(19) United States
(12) Patent Application Publication
(10) Pub. No.: US 2022/0096609 A1
(43) Pub. Date: Mar. 31, 2022

(54) COMBINATION THERAPY USING CLOSTRIDIAL TOXIN DERIVATIVE AND AT LEAST ONE CHEMICAL DEPOLARIZING AGENT

(71) Applicant: Allergan, Inc., Irvine, CA (US)

(72) Inventors: James Oliver Dolly, Dublin (IE); Tomas Zurawski, Dublin (IE); Ron Broide, San Marcos, CA (US); Amy Brideau-Andersen, San Clemente, CA (US); James Cunningham, San Clemente, CA (US); Gregory Nicholson, Cypress, CA (US)

(21) Appl. No.: 17/429,237

(22) PCT Filed: Feb. 5, 2020
(86) PCT No.: PCT/US2020/016824

§ 371 Date: Aug. 6, 2021

Related U.S. Application Data
(60) Provisional application No. 62/801,699, filed on Feb. 6, 2019.

(51) Int. Cl. A61K 38/48; A61K 47/18; A61K 45/06; C12N 9/64

(52) U.S. Cl. CPC A61K 38/4893; A61K 47/18; C12Y 304/24069; C12N 9/6489; A61K 45/06

(57) ABSTRACT

Formulations, methods, and kits comprising at least one Clostridium toxin derivative and at least one chemical depolarizing agent suitable for inducing local, partial or complete muscle paralysis or muscle denervation in a subject are described.

### CROSS-REFERENCE TO RELATED APPLICATIONS

This application claims priority to U.S. Provisional Application No. 62/801,699, filed Feb. 6, 2019, the entire content of which is incorporated herein by reference.

### TECHNICAL FIELD

The subject matter described herein relates to combination therapy using Clostridial toxin and derivatives thereof and one or more chemical depolarizing agents.

### BACKGROUND

The anaerobic, gram positive bacterium Clostridum botulinum produces a potent polypeptide neurotoxin, botulinum toxin, which causes a neuroparalytic illness in humans and animals referred to as botulism. The spores of Clostridum botulinum are found in soil and can grow in improperly sterilized and sealed food containers of home-based canneries, which are the cause of many of the cases of botulism. The effects of botulism typically appear 18to 36 hours after eating the foodstuffs infected with a Clostridum botulinum culture or spores. The botulinum toxin can apparently pass unattenuated through the lining of the gut and shows a high affinity for cholinergic motor neurons. Symptoms of botulinum toxin intoxication can progress from difficulty walking, swallowing and speaking to paralysis of the respiratory muscles and death. Aminopyridines have been found to rescue BoNT/A intoxicated mouse phrenic nerve-hemidiaphragm cells (Mayorov et al., ACS Chem Bio, 5(12):1183-1191 (2010)).

Commercially available botulinum toxin containing pharmaceutical compositions include BOTOX® (botulinum toxin type A neurotoxin complex with human serum albumin and sodium chloride) (Allergan, Inc., Irvine, Calif.), DYSPORT® (Clostridum botulinum type A toxin haemagglutinin complex with human serum albumin and lactose in the formulation) (Ipsen Limited, Berkshire, U.K.) as a powder to be reconstituted with 0.9% sodium chloride before use), XEOMIN® (botulinum toxin type A neurotoxin without accessory proteins, human albumin, and sucrose)(Merz Pharmaceuticals GmbH, Frankfort, Germany) as a powder to be reconstituted with 0.9% sodium chloride before use, JEUVEAU™ (botulinum toxin type A)(Evolus, Inc., Santa Barbara, Calif.) and MYOBLOC™ (an injectable solution comprising botulinum toxin type B, human serum albumin, sodium succinate, and sodium chloride at about pH 5.6 (Solstice Neurosciences, Inc., South San Francisco, Calif.). More recently, non-protein-based (animal protein-free) botulinum toxin containing pharmaceutical compositions have been described (see, e.g. U.S. Published Application No. 2012/0207787, the contents of which are incorporated by reference herein). Exemplary compositions described include any of the Clostridial neurotoxin actives and one or more excipients including a sugar and/or a surfactant.

Botulinum toxin is a large protein for incorporation into a pharmaceutical formulation (the molecular weight of the botulinum toxin type A complex is 900 kD) and is inherently fragile and labile. The size of the toxin complex makes it much more friable and labile than smaller, less complex proteins, thereby compounding the formulation and handling difficulties if botulinum toxin stability is to be maintained. Hence, a botulinum toxin stabilizer must be able to interact with the toxin in a manner which does not denature, fragment or otherwise detoxify the toxin molecule or cause disassociation of the non-toxin proteins present in the toxin complex.

Botulinum toxins have been used for various therapeutic and cosmetic purposes including treating cervical dystonia, blepharospasm, strabismus, spasticity, headache, hyperhidrosis, overactive bladder, rhinitis, bruxism, enlarged prostate, achalasia, anismus, sphincter of Oddi malfunction, acne, tremors, juvenile cerebral palsy, and facial wrinkles.

As the most lethal known biological product, exceptional safety, precision, and accuracy are called for at all steps of the formulation of a botulinum toxin containing pharmaceutical composition.

A botulinum toxin pharmaceutical composition that provides an extended or sustained release while maintaining safety and accuracy is desired.

### BRIEF DESCRIPTION OF THE DRAWINGS

FIG. 1 is a graph showing the average DAS per day in rats after administration of a botulinum toxin type A formulated in saline as compared to a botulinum toxin type A formulated in 100 μM 4-AP.

FIGS. 2A-2B are a graph showing the normalized muscle tension (%) per minute after administration of 4-AP with BoNT/A and a graph showing the time required for 50% paralysis after administration of 300 μM 4-AP or saline followed by 30 pM BoNT/A.

FIGS. 3A-3B are graphs showing the normalized muscle tension (%) per minute after administration of DAP with BoNT/E (100 pM in FIG. 3A and 10 pM in FIG. 3B) and BoNT/E alone (100 pM in FIG. 3A and 10 pM in FIG. 3B), respectively.

FIGS. 4A-4B are graphs showing DAP-increased uptake of BoNT/E (100 pM in FIG. 4A and 10 pM in FIG. 4B) leading to faster onset of neuroparalysis in mouse.

### BRIEF SUMMARY

The following aspects and embodiments thereof described and illustrated below are meant to be exemplary and illustrative, not limiting in scope.

In some aspects methods, formulations, systems, and kits are provided comprising a therapeutically effective amount of a Clostridial toxin derivative and at least one chemical depolarizing agent to accelerate onset and to extend therapeutic duration of the Clostridial toxin derivative.

In one aspect a method of inducing local, partial or complete muscle denervation in a subject by administering each of a therapeutically effective amount of a Clostridial toxin derivative and at least one chemical depolarizing agent is provided where the at least one chemical depolarizing agent is administered within about for example, one week, such as within about 24 hours of administering the Clostridial toxin derivative. In embodiments, the Clostridial toxin derivative and depolarizing agent are administered at the same time. In some embodiments, the Clostridial toxin derivative and depolarizing agent are administered in the same composition.

In embodiments, the at least one chemical depolarizing agent is selected from a potassium channel blocker, a calcium channel ionophore, sodium channel ionophore, and potassium. In one embodiment, at least one of the at least one chemical depolarizing agents is 4-aminopyridine (4-AP). In some embodiments, at least one of the at least one chemical depolarizing agents is 3,4-diaminopyridine (DAP).

In embodiments, the Clostridial toxin derivative is a botulinum toxin. In some embodiments, the botulinum toxin is selected from the group consisting of botulinum toxin types BoNT/A, BoNT/B, BoNT/C, BoNT/D, BoNT/E, BoNT/F, BoNT/G, BoNT/H, BoNT/X and eBoNT/J. In some embodiments, the botulinum toxin is selected from the group consisting of mosaic toxins. Examples of mosaic toxins include BoNT/DC, BoNT/CD, and BoNT/FA. In some embodiments, about 1-200 Units of the Clostridial toxin derivative is administered.

In embodiments, administering the at least one chemical depolarizing agent extends the duration of muscle denervation in the subject as compared to administering the Clostridial toxin derivative alone. In some embodiments, the extended duration of muscle denervation is at least about 50-200% longer as compared to administering the Clostridial toxin derivative alone. In some embodiments, the duration of muscle denervation is at least about 1-14 days longer than the duration of muscle denervation when the Clostridial toxin derivative is administered alone. In some embodiments, the therapeutically effective amount of the Clostridial toxin derivative administered in the method is lower than that of the Clostridial toxin derivative administered alone.

In some embodiments, administering the at least one chemical depolarizing agent enhances neurotransmission followed by a faster muscle denervation in the subject as compared to administering the Clostridial toxin derivative alone. In some embodiments, administering DAP enhances neurotransmission followed by a faster muscle denervation in the subject as compared to administering the Clostridial toxin derivative alone. In some embodiments, administering DAP enhances neurotransmission followed by a faster muscle denervation in the subject as compared to administering BoNT/E alone. In some embodiments, the therapeutically effective amount of the Clostridial toxin derivative administered in the method is lower than that of the Clostridial toxin derivative administered alone.

In embodiments, at least one of the Clostridial toxin derivative and the depolarizing agent are locally administered. In embodiments, at least one of the Clostridial toxin derivative and the depolarizing agent are administered by injection. In some embodiments, administration by injection is selected from the group consisting of non-intramuscular injection and subdermal injection. In embodiments, at least one of the Clostridial toxin derivative and at least one of the depolarizing agents are administered by intramuscular, non-intramuscular, intraarticular, extraarticular, intradermal, subcutaneous administration, intrathecal administration, intraperitoneal administration, implantation (for example, of a slow-release device such as polymeric implant or miniosmotic pump), or combinations thereof.

In embodiments, administering the Clostridial toxin derivative and the depolarizing agent to induce local, partial or complete muscle denervation is effective to treat a condition or symptom selected from the group consisting of a neuromuscular disease, pain, a urological disorder, inflammation, bacterial infection, wound healing, scarring and skin disorders. In some embodiments, inducing local, partial or complete muscle denervation is used for cosmetically modifying soft-tissue features of the subject.

In another aspect a method of inducing local, partial or complete muscle paralysis in a subject by administering each of a therapeutically effective amount of a Clostridial toxin derivative and at least one chemical depolarizing agent is provided where the at least one chemical depolarizing agent is administered within for example about one week, such as within about 24 hours of administering the Clostridial toxin derivative. In embodiments, the Clostridial toxin derivative and depolarizing agent are administered at the same time. In some embodiments, the Clostridial toxin derivative and depolarizing agent are administered in the same composition.

In embodiments, the at least one chemical depolarizing agent is selected from a potassium channel blocker, a calcium channel ionophore, a sodium channel ionophore, and potassium. In one embodiment, at least one of the at least one chemical depolarizing agents is 4-aminopyridine (4-AP). In one embodiment, at least one of the at least one chemical depolarizing agents is DAP.

In embodiments, the Clostridial toxin derivative is a botulinum toxin. In some embodiments, the botulinum toxin is selected from the group consisting of botulinum toxin types BoNT/A, BoNT/B, BoNT/C, BoNT/D, BoNT/E, BoNT/F, BoNT/G, BoNT/H, BoNT/X and eBoNT/J. In some embodiments, the botulinum toxin is selected from the group consisting of mosaic toxins. Examples of mosaic toxins include BoNT/DC, BoNT/CD, and BoNT/FA. In some embodiments, about 1-200 Units of the Clostridial toxin derivative is administered.

In embodiments, administering the at least one chemical depolarizing agent extends the duration of muscle paralysis in the subject as compared to administering the Clostridial toxin derivative alone. In some embodiments, the increase in duration of muscle paralysis is at least about 50-200% as compared to administering the Clostridial toxin derivative alone. In some embodiments, the duration of muscle paralysis is at least about 1-14 days longer than the duration of muscle paralysis when the Clostridial toxin derivative is administered alone.

In embodiments, at least one of the Clostridial toxin derivative and the depolarizing agent are locally administered. In embodiments, at least one of the Clostridial toxin derivative and the depolarizing agent are administered by injection. In some embodiments, administration by injection is selected from the group consisting of non-intramuscular injection and subdermal injection.

In some embodiments, administering the at least one chemical depolarizing agent enhances neurotransmission followed by a faster muscle denervation in the subject as compared to administering the Clostridial toxin derivative alone. In some embodiments, administering DAP enhances neurotransmission followed by a faster muscle denervation in the subject as compared to administering the Clostridial toxin derivative alone. In some embodiments, administering DAP enhances neurotransmission followed by a faster muscle denervation in the subject as compared to administering BoNT/E alone. In some embodiments, the therapeutically effective amount of the Clostridial toxin derivative administered in the method is lower than that of the Clostridial toxin derivative administered alone.

In embodiments, administering the Clostridial toxin derivative and the depolarizing agent to induce local, partial or complete muscle paralysis is effective to treat a condition or symptom selected from the group consisting of a neuromuscular disease, pain, a urological disorder, inflammation, and skin disorders. In some embodiments, inducing local, partial or complete muscle paralysis is used for cosmetically modifying soft-tissue features of the subject.

In another aspect, a pharmaceutical preparation is provided. The preparation comprises a therapeutically effective amount of a Clostridial toxin derivative and at least one chemical depolarizing agent

In another aspect, a pharmaceutical preparation for use in inducing local, partial or complete muscle denervation in a subject is provided. The preparation comprises a therapeutically effective amount of a Clostridial toxin derivative and at least one chemical depolarizing agent.

In embodiments, the at least one chemical depolarizing agent is selected from a potassium channel blocker, a calcium channel ionophore, a sodium channel ionophore, and potassium. In some embodiments, at least one of the chemical depolarizing agents is 4-aminopyridine (4-AP). In some embodiments, at least one of the chemical depolarizing agents is DAP. In some embodiments, at least one of the chemical depolarizing agents is a calcium channel ionophore selected from the group consisting of ionmycin and calcimycin. In some embodiments, at least one of the chemical depolarizing agents is a sodium channel ionophore selected from the group consisting of monensin and gramicidin.

In embodiments, the Clostridial toxin derivative is a botulinum toxin. In some embodiments, the botulinum toxin is selected from the group consisting of botulinum toxin types BoNT/A, BoNT/B, BoNT/C, BoNT/D, BoNT/E, BoNT/F, BoNT/G, BoNT/H, BoNT/X and eBoNT/J. In some embodiments, the botulinum toxin is selected from the group consisting of mosaic toxins. Examples of mosaic toxins include BoNT/DC, BoNT/CD, and BoNT/FA. In some embodiments, the therapeutically effective amount of the Clostridial toxin derivative is about 1-200 Units.

In embodiments, the preparation further comprises at least one stabilizer. In some embodiments, the at least one stabilizer is selected from an albumin, a non-oxidizing amino acid derivative, a caprylate, a polysorbate, an amino acid, and a divalent metal cation.

In another aspect, a pharmaceutical preparation for use in inducing local, partial or complete muscle paralysis in a subject is provided. The preparation comprises a therapeutically effective amount of a Clostridial toxin derivative and at least one chemical depolarizing agent.

In embodiments, the at least one chemical depolarizing agent is selected from a potassium channel blocker, a calcium channel ionophore, a sodium channel ionophore, and potassium. In some embodiments, at least one of the chemical depolarizing agents is 4-aminopyridine (4-AP). In some embodiments, at least one of the chemical depolarizing agents is DAP. In some embodiments, at least one of the chemical depolarizing agents is a calcium channel ionophore selected from the group consisting of ionmycin and calcimycin. In some embodiments, at least one of the chemical depolarizing agents is a sodium channel ionophore selected from the group consisting of monensin and gramicidin.

In embodiments, the at least one chemical depolarizing agent is selected from a potassium channel blocker, a calcium channel ionophore, a sodium channel ionophore, and potassium. In some embodiments, at least one of the chemical depolarizing agents is 4-aminopyridine (4-AP). In some embodiments, at least one of the chemical depolarizing agents is DAP. In some embodiments, at least one of the chemical depolarizing agents is a calcium channel ionophore selected from the group consisting of ionmycin and calcimycin. In some embodiments, at least one of the chemical depolarizing agents is a sodium channel ionophore selected from the group consisting of monensin and gramicidin.

In embodiments, the Clostridial toxin derivative is a botulinum toxin. In some embodiments, the botulinum toxin is selected from the group consisting of botulinum toxin types BoNT/A, BoNT/B, BoNT/C, BoNT/D, BoNT/E, BoNT/F, BoNT/G, BoNT/H, BoNT/X and eBoNT/J. In some embodiments, the botulinum toxin is selected from the group consisting of mosaic toxins. Examples of mosaic toxins include BoNT/DC, BoNT/CD, and BoNT/FA. In some embodiments, the therapeutically effective amount of the Clostridial toxin derivative is about 1-200 Units.

In embodiments, the preparation further comprises at least one stabilizer. In some embodiments, the at least one stabilizer is selected from an albumin, a non-oxidizing amino acid derivative, a caprylate, a polysorbate, an amino acid, and a divalent metal cation.

In embodiments of the preparation of each aspect, the preparation does not comprise a tissue, organ, or cells. In other words, the preparation is not formed in situ during an in vitro or in vivo study involving a tissue, organ, cell or subject.

In embodiments of preparation of each aspect, the preparation does not comprise an additional active ingredient other than the Clostridial toxin derivative and the at least one chemical depolarizing agent.

In a further aspect, a method of inducing local, partial or complete muscle denervation in a subject is provided. The method comprises providing for administration a therapeutically effective amount of a Clostridial toxin derivative; and providing for administration, or instructing to administer, a chemical depolarizing agent. The chemical depolarizing agent is administered, or instructed to be administered, within for example, about one week, such as within about 24 hours of administration of the Clostridial toxin derivative, which induces local, partial or complete muscle denervation in the subject.

In yet another aspect, a method of inducing local, partial or complete muscle paralysis is provided. The method comprises providing for administration a therapeutically effective amount of a Clostridial toxin derivative; and providing for administration, or instructing to administer, a chemical depolarizing agent. The chemical depolarizing agent is administered, or instructed to be administered, within for example, about one week, such as within about 24 hours of administration of the Clostridial toxin derivative, which induces local, partial or complete muscle paralysis in the subject.

In a further aspect, a method of inducing local, partial or complete muscle paralysis or muscle denervation in a subject is provided. The method comprises providing for administration a therapeutically effective amount of a Clostridial toxin derivative; and instructing administration of a chemical depolarizing agent within for example about one week, such as within about 24 hours of administration of the Clostridial toxin derivative, whereby administration of the chemical depolarizing agent as instructed provides local, partial or complete muscle paralysis or muscle denervation in the subject.

In yet another aspect, a method of inducing local, partial or complete muscle paralysis or muscle denervation in a subject is provided. The method comprises providing for administration a chemical depolarizing agent; and instructing administration of a therapeutically effective amount of a Clostridial toxin derivative within for example about one week, such as within about 24 hours of administration of the chemical depolarizing agent, whereby administration of the Clostridial toxin derivative as instructed provides local, partial or complete muscle paralysis or muscle denervation in the subject.

Certain compositions described herein provide stable liquid or solid pharmaceutical composition comprising a Clostridial toxin derivative. The compositions may further comprise a disaccharide, a surfactant, and/or an antioxidant. The pharmaceutical compositions can be applied topically. The pharmaceutical compositions can also be in the form of microneedle patches.

Certain embodiments also provide methods for the treatment of various diseases, disorders, and conditions, including, for example, depression, headache (such as, for example, migraine, tension headache, and the like), pain, hyperhidrosis, muscle spasticity, cervical dystonia, blepharospasm, overactive bladder (neurogenic detrusor overactivity, and idiopathic overactive bladder), bacterial infections, wound healing, scars, skin conditions including, for example, wrinkles, irregularities, depressions, and the like using the compositions provided. Embodiments can include various administration techniques, including, for example, injection, such as intramuscular, intracutaneous or subcutaneous injection, or the like, instillation, intravenous, transdermal, and topical. In some embodiments, at least the Clostridial toxin derivative is administered by a non-intramuscular injection. In some embodiments, at least the Clostridial toxin derivative is administered by subdermal injection.

In addition to the exemplary aspects and embodiments described above, further aspects and embodiments will become apparent by reference to the examples and by study of the following descriptions.

Additional embodiments of the present methods and compositions, and the like, will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment herein. Additional aspects and advantages of the present compositions and methods are set forth in the following description and claims, particularly when considered in conjunction with the accompanying examples.

### DETAILED DESCRIPTION

### I. DEFINITIONS

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 82 m are also explicitly disclosed, as well as the range of values greater than or equal to 1 82 m and the range of values less than or equal to 8 μm.

The singular forms “a,” “an,” and “the” include plural referents unless the context clearly dictates otherwise. Thus, for example, reference to an “excipient” includes a single excipient as well as two or more of the same or different excipients, and the like.

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. In some embodiments, “about” means that the item, parameter or term so qualified encompasses a range of plus or minus ten percent above and/or below the value of the stated item, parameter or term.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Animal protein free” means the absence of blood derived, blood pooled and other animal derived products or compounds. “Animal” means a mammal (such as a human), bird, reptile, fish, insect, spider or other animal species. “Animal” excludes microorganisms, such as bacteria. Thus, an animal protein free pharmaceutical composition can include a botulinum neurotoxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises or which consists of a botulinum toxin (as the active ingredient) and a suitable polysaccharide as a stabilizer or excipient.

“Biological activity” describes the beneficial or adverse effects of a drug on living matter. When a drug is a complex chemical mixture, this activity is exerted by the substance's active ingredient but can be modified by the other constituents. Biological activity can be assessed as potency or as toxicity by an in vivo LD₅₀ or ED₅₀ assay, or through an in vitro assay such as, for example, cell-based potency assays as described in U.S. 2010/0203559 and U.S. 2010/0233802.

“Botulinum toxin” means a neurotoxin produced by Clostridum botulinum, as well as a botulinum toxin (or the light chain or the heavy chain thereof) made recombinantly by a non-Clostridial species. The phrase “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a BoNT/H, BoNT/X, eBoNT/J, mosaic toxins (such as BoNT/DC, BoNT/CD, and BoNT/FA), a Tetanus toxin (TeNT), a Baratii toxin (BaNT), and a Butyricum toxin (BuNT). The BoNT/C2 cytotoxin and BoNT/C3 cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Baratii toxin complex, and a Butyricum toxin complex.0 Non-limiting examples of Clostridial toxin complexes include those produced by a Clostridum botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a Clostridial toxin that exerts an effect upon or after administration to a subject or patient. As used herein, the term “Clostridial toxin active ingredient” encompasses a Clostridial toxin complex comprising the approximately 150-kDa Clostridial toxin and other proteins collectively called non-toxin associated proteins (NAPs), the approximately 150-kDa Clostridial toxin alone, or a modified Clostridial toxin, such as, e.g., a re-targeted Clostridial toxins.

A “Clostridial toxin derivative” as used herein refers to a “Clostridial toxin” as described above as well as a modified, recombinantly produced, or fragment of a Clostridial toxin.

“Deformity” means a cosmetic, physical or functional irregularity, defect, abnormality, imperfection, malformation, depression, or distortion.

“Effective amount” as applied to the biologically active ingredient means that amount of the ingredient which is generally sufficient to effect a desired change in the subject. For example, where the desired effect is a reduction in an autoimmune disorder symptom, an effective amount of the ingredient is that amount which causes at least a substantial reduction of the autoimmune disorder symptom, and without resulting in significant toxicity.

“Effective amount” when used in reference to the amount of an excipient or specific combination of excipients added to a Clostridial toxin composition, refers to the amount of each excipient that is necessary to achieve the desired initial recovered potency of a Clostridial toxin active ingredient. In aspects of this embodiment, an effective amount of an excipient or combination of excipients results in an initial recovered potency of, e.g., at least 10%, at least 20%, at least 30%, at least 40%, at least 50%, at least 60%, at least 70%, at least 80%, at least 90% or at least 100%. In other aspects of this embodiment, a therapeutically effective concentration of a Clostridial toxin active ingredient reduces a symptom associated with the aliment being treated by, e.g., at most 10%, at most 20%,at most 30%, at most 40%, at most 50%, at most 60%, at most 70%, at most 80%, at most 90% or at most 100%.

“Heavy chain” means the heavy chain of a botulinum neurotoxin. It has a molecular weight of about 100 kDa and can be referred to as the H chain, or as H.

H_{C} means a fragment (about 50 kDa) derived from the H chain of a botulinum neurotoxin which is approximately equivalent to the carboxyl end segment of the H chain, or the portion corresponding to that fragment in the intact H chain. It is believed to be immunogenic and to contain the portion of the natural or wild type botulinum neurotoxin involved in high affinity, presynaptic binding to motor neurons.

H_{N} means a fragment (about 50 kDa) derived from the H chain of a botulinum neurotoxin which is approximately equivalent to the amino end segment of the H chain, or the portion corresponding to that fragment in the intact in the H chain. It is believed to contain the portion of the natural or wild type botulinum neurotoxin involved in the translocation of the L chain across an intracellular endosomal membrane.

“Implant” means a controlled or sustained release (e.g., pulsatile or continuous) composition or drug delivery system. The implant can be, for example, injected, inserted or implanted into a human body and may take a solid, particulate, gel, liquid or foam form amongst others.

“Light chain” means the light chain of a Clostridial neurotoxin. It has a molecular weight of about 50 kDa, and can be referred to as the L chain, L, or as the proteolytic domain (amino acid sequence) of a botulinum neurotoxin.

LH_{N} or L-H_{N} means a fragment derived from a Clostridial neurotoxin that contains the L chain, or a functional fragment thereof coupled to the H_{N} domain. It can be obtained from the intact Clostridial neurotoxin by proteolysis, so as to remove or to modify the H_{C} domain.

“Implant” means a controlled release (e.g., pulsatile or continuous) composition or drug delivery system. The implant can be, for example, injected, inserted or implanted into a human body.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter.

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

“Patient” means a human or non-human subject receiving medical or veterinary care. Accordingly, the compositions as disclosed herein can be used in treating any animal, such as, for example, mammals, or the like.

“Peripherally administering” or “peripheral administration” means subdermal, intradermal, transdermal, or subcutaneous administration, but excludes intramuscular administration. “Peripheral” means in a subdermal location and excludes visceral sites.

“Pharmaceutical composition” means a composition comprising an active pharmaceutical ingredient, such as, for example, a Clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to a subject and encompasses compounds such as, e.g., a stabilizing agent, a bulking agent, a cryo-protectant, a lyo-protectant, an additive, a vehicle, a carrier, a diluent, or an auxiliary. An excipient generally is mixed with an active ingredient or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two (or multiple) component system, for example a vacuum-dried composition reconstituted with a reconstitution vehicle which can, for example, contain an ingredient not present in the initial compounding of the pharmaceutical composition. A multiple component system can provide several benefits, including that of allowing incorporation of ingredients which are not sufficiently compatible for long-term shelf storage with the first component of the two component system. For example, the reconstitution vehicle may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (e.g. in the reconstitution vehicle) at the approximate time of use. A multiple component system may also allow separate administration of ingredients of a formulation. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

“Polysaccharide” means a polymer of more than two saccharide molecule monomers. The monomers can be identical or different.

“Stabilizing agent”, “stabilization agent” or “stabilizer” means a substance that acts to stabilize a Clostridial toxin active ingredient such that the potency of the pharmaceutical composition is increased relative to an unstabilized composition.

“Stabilizers” can include excipients, and can include protein and non-protein molecules. Examples of stabilizers include, but are not limited to, surfactants, polymers, polyols, a poloxamer, albumin (i.e. human serum albumin), gelatin, trehalose, proteins, sugars, polyvinylpyrrolidone, N-acetyl-tryptophan (“NAT”), caprylate (i.e. sodium caprylate), a polysorbate (i.e. P80), amino acids, and divalent metal cations such as zinc. Stabilizers may also include a non-oxidizing amino acid derivative or an amino acid.

“Stabilizing”, “stabilizes”, or “stabilization” mean that an active pharmaceutical ingredient (“API”) retains at least about 20% and up to about 100% of its biological activity (which can be assessed as potency or as toxicity by an in vivo LD₅₀ or ED₅₀ measure) in the presence of a compound which is stabilizing, stabilizes or which provides stabilization to the API as compared to the potency or toxicity of the biologically active botulinum toxin prior to being incorporated into the pharmaceutical composition.

“Substantially” or “essentially” means nearly totally or completely, for instance, 90-95% or greater of some given quantity.

“Substantially free” means nearly totally or completely absent of some given quantity such as being present at a level of less than about 1-5 of some given quantity. In some embodiments, “substantially free” means presence at a level of less than or equal to 1-5% by weight of the pharmaceutical composition.

“Sustained release” means release of an active agent (such as a botulinum toxin) over a period of about seven days or more, while “extended release” means release of an active agent over a period of time of less than about seven days.

“Therapeutic formulation” means a formulation can be used to treat and thereby alleviate a disorder or a disease, such as, for example, a disorder or a disease characterized by hyperactivity (i.e. spasticity) of a peripheral muscle.

“TEM” as used herein, is synonymous with “Targeted Exocytosis Modulator” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Tonicity agent” means a low molecular weight excipient which is included in a formulation to provide isotonicity. Disaccharides, such as trehalose or sucrose, polyalcohols, such as sorbitol or mannitol, monosaccharides, such as glucose, and salts, such as sodium chloride, can serve as a tonicity agent.

“Topical administration” excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Treating” means to alleviate (or to eliminate) at least one symptom of a condition or disorder, such as, for example, wrinkles, spasticity, depression, pain (such as, for example, headache pain), bladder overactivity, or the like, either temporarily or permanently.

“Variant” means a Clostridial neurotoxin, such as wild-type botulinum toxin serotype BoNT/A, BoNT/B, BoNT/C, BoNT/D, BoNT/E, BoNT/F, BoNT/G, BoNT/H, BoNT/X, eBoNT/J, and mosaic toxins such as BoNT/DC, BoNT/CD, and BoNT/FA, that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell. An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased compared to the parent chemical entity. For example, a variant light chain of a botulinum toxin type A having an amino acid sequence removed may have a shorter biological persistence than that of the parent (or native) botulinum toxin type A light chain.

### II. COMBINATION THERAPY

In one aspect a treatment, composition, formulation or system for inducing local, partial, or complete muscle denervation or paralysis in a subject is provided. The treatment comprises administering a combination of at least one Clostridial toxin derivative and at least one chemical depolarizing agent.

In some embodiments, the Clostridial toxin derivative includes a native, recombinant Clostridial toxin, recombinant modified toxin, fragments thereof, targeted exocytosis modulators (TEMs), or combinations thereof. In some embodiments, the Clostridial derivative is a botulinum toxin. In alternative embodiments, the Clostridial derivative is a TEM. In some embodiments, the Clostridial derivative is a stapled toxin as described in J. Neurochem., 2013 July; 126(2): 223-33. Doi:10.1111/jnc. 12284. Epub 2013 May 20, which is herein incorporated by reference.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

Several generally immunologically distinct botulinum neurotoxins have been characterized, these being respectively botulinum neurotoxin serotypes BoNT/A, BoNT/B, BoNT/C, BoNT/D, BoNT/E, BoNT/F, BoNT/G, BoNT/H, BoNT/X and eBoNT/J, and mosaic toxins such as BoNT/DC, BoNT/CD, and BoNT/FA each of which is distinguished by neutralization with type-specific antibodies. The different serotypes of botulinum toxin vary in the animal species that they affect and in the severity and duration of the paralysis they evoke. Botulinum toxin apparently binds with high affinity to cholinergic motor neurons, is translocated into the neuron and blocks the release of acetylcholine. In some embodiments, the Clostridial toxin derivative is a botulinum toxin, which is selected from the group consisting of botulinum toxin types A, B, C (C₁), D, E, F and G. In one embodiment, the Clostridial derivative of the present method is a botulinum toxin type A (BoNT/A). In one embodiment, the Clostridial derivative of the present method is a botulinum toxin type E (BoNT/E). The botulinum toxin can be a recombinant botulinum neurotoxin, such as botulinum toxins produced by E. coli.

The molecular weight of the neurotoxic component of a botulinum toxin complex is about 150 kD. Botulinum toxin is typically made by the Clostridial botulinum bacterium as a complex comprising the 150 kD botulinum toxin protein molecule and associated non-toxin proteins. Thus, a botulinum toxin type A complex can be produced by Clostridial bacterium as 900 kD, 500 kD and 300 kD complex forms.

The Clostridial toxin derivative, such as a botulinum toxin, for use as described herein can be stored in lyophilized, vacuum dried form in containers under vacuum pressure or as stable liquids. Prior to lyophilization the botulinum toxin can be combined with one or more pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. Non-protein excipients are described, for example, in U.S. Pat. No. 8,137,677, which is incorporated by reference herein in its entirety. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the patient.

In some embodiments, to increase the resident time of the Clostridial toxin derivative after administration, the Clostridial toxin derivative is provided in a controlled release system comprising a polymeric matrix encapsulating the Clostridial toxin derivative, wherein fractional amount of the Clostridial toxin derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The therapeutically effective amount of the Clostridial toxin derivative, for example a botulinum toxin, administered according to the present method can vary according to the potency of the toxin and particular characteristics of the condition being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18to 20 female Swiss-Webster mice, weighing about 20 grams each.

The therapeutically effective amount of the botulinum toxin according to the present method can vary according to the potency of a particular botulinum toxin, as commercially available Botulinum toxin formulations do not have equivalent or interchangeable potency units. For example, one unit of BOTOX (onabotulinumtoxinA), a botulinum toxin type A available from Allergan, Inc., may in some cases have a potency unit that is approximately equal to 3 to 5 units of DYSPORT® (abobotulinumtoxinA), also a botulinum toxin type A available from Ipsen Pharmaceuticals. In some embodiments, the amount of abobotulinumtoxinA, (such as DYSPORT®), administered in the present method is about three to four times the amount of onabotulinumtoxinA (such as BOTOX®) administered, as comparative studies have suggested that one unit of onabotulinumtoxinA may in some cases have a potency that is approximately equal to three to four units of abobotulinumtoxinA. MYOBLOC®, a botulinum toxin type B available from Elan, has a much lower potency unit relative to BOTOX®. In some embodiments, the botulinum neurotoxin can be a pure toxin, devoid of complexing proteins, such as XEOMIN® (incobotulinumtoxinA). One unit of incobotulinumtoxinA has potency approximately equivalent to one unit of onabotulinumtoxinA. The quantity of toxin administered and the frequency of its administration will be at the discretion of the physician responsible for the treatment and will be commensurate with questions of safety and the effects produced by a particular toxin formulation.

In embodiment, a therapeutically effective amount of the Clostridial toxin derivative may vary depending upon the condition being treated. In some embodiments, a therapeutically effective amount of the Clostridial toxin derivative comprises about 1-200 Units.

At least one chemical depolarizing agent is administered in combination with the Clostridial toxin derivative. In some embodiments, the at least one chemical depolarizing agent is administered in combination with the Clostridial toxin derivative in order to increase duration of the therapeutic effect of the Clostridial toxin derivative. In some embodiments, the at least one chemical depolarizing agent is administered in combination with the Clostridial toxin derivative in order to accelerate onset of the Clostridial toxin derivative therapeutic effect such as intoxication. Previous studies have used potassium channel blockers to reverse BoNT-induced inhibition of Ach release (see, e.g. Sterling et al., Soc Neurosci Abstr, 1993, 19(1-3):1764, abstract). However, it has surprisingly been found that a combination therapy comprising Clostridial toxin derivative and at least one chemical depolarizing agent is effective to increase the duration and speed of intoxication of therapeutic efficacy of the Clostridial toxin derivative. In embodiments, combined administration (administered together or within for example about one week, such as within about 24 hours of each other) of a Clostridial toxin derivative and a chemical depolarizing agent is effective to increase the duration of effect on local, partial or complete muscle denervation in a subject as compared to administration of the Clostridial toxin derivative alone. In embodiments, combined administration of a Clostridial toxin derivative and a chemical depolarizing agent is effective to increase the duration of effect of local, partial or complete muscle paralysis in a subject as compared to administration of the Clostridial toxin derivative alone. In some embodiments, combined administration of a Clostridial toxin derivative and a chemical depolarizing agent is effective to increase the time for the effect of local, partial or complete muscle paralysis in a subject as compared to administration of the Clostridial toxin derivative alone (e.g. shorten the time for the Clostridial toxin derivative to become effective).

In some embodiments, combined administration of a Clostridial toxin derivative and a chemical depolarizing agent enhances neurotransmission followed by a faster muscle denervation in the subject as compared to administering the Clostridial toxin derivative alone. In some embodiments, combined administration of a Clostridial toxin derivative and DAP enhances neurotransmission followed by a faster muscle denervation in the subject as compared to administering the Clostridial toxin derivative alone. In some embodiments, combined administration of BoNT/E and DAP enhances neurotransmission followed by a faster muscle denervation in the subject as compared to administering BoNT/E alone. In some embodiments, combined administration of a Clostridial toxin derivative and DAP reduces the therapeutically effective amount of the Clostridial toxin derivative by about 25-600% as compared to the therapeutically effective amount of the Clostridial toxin derivative administered alone. In some embodiments, combined administration of BoNT/E and DAP reduces the therapeutically effective amount of BoNT/E by about 25-600% as compared to the therapeutically effective amount of BoNT/E administered alone.

In embodiments, combined administration of a Clostridial toxin derivative and at least one chemical depolarizing agent is effective to increase the duration of effect (muscle denervation and/or paralysis) by at least about 25-200% as compared to administering the Clostridial toxin derivative in the absence of the chemical depolarizing agent. The effect of administering the Clostridial toxin derivative may be measured by any suitable means in the art. In some embodiments, the effect of administering the Clostridial toxin derivative is measured using a digital abduction assay score (see, e.g. Broide, et al., Toxicon, 2013, 71:18-24). In some embodiments, the combined administration is effective to increase the duration of the effect at least about 25-175%, at least about 25-150%, at least about 25-125%, at least about 25-100%, at least about 25-75%, at least about 25-50%, at least about 50-200%, at least about 50-175%, at least about 50-150%, at least about 50-125 %, at least about 50-100%, at least about 50-75%, at least about 75-200%, at least about 75-175%, at least about 75-150%, at least about 75-125 %, at least about 75-100%, at least about 100-200%, at least about 100-175%, at least about 100-150%, at least about 100-125 %, at least about 125-200%, at least about 125-175%, at least about 125-150%, at least about 150-200%, at least about 150-175%, or at least about 175-200% as compared to administering the Clostridial toxin derivative in the absence of the chemical depolarizing agent. In embodiments, the combined administration is effective to increase duration of the effect at least about 25%, at least about 50%, at least about 75%, at least about 100%, at least about 125 %, at least about 150%, at least about 175%, at least about 200% or more as compared to administering the Clostridial toxin derivative in the absence of the chemical depolarizing agent. In embodiments, the combined administration is effective to increase the duration of effect up to about 25%, up to about 50%, up to about 75%, up to about 100%, up to about 125 %, up to about 150%, up to about 175%, up to about 200% as compared to administering the Clostridial toxin derivative in the absence of the chemical depolarizing agent.

In some embodiments, combined administration of a Clostridial toxin derivative and at least one chemical depolarizing agent is effective to increase the duration of effect (muscle denervation and/or paralysis) by at least about 1-30 days as compared to administering the Clostridial toxin derivative in the absence of the chemical depolarizing agent. In embodiments, the combined administration is effective to increase the duration of effect by about 1-28 days, about 1-21 days, about 1-20 days, about 1-15 days, about 1-14 days, about 1-10 days, about 1-7 days, about 1-5 days, about 1-3 days, about 1-2 days, about 2-30 days, about 2-28 days, about 2-21 days, about 2-20 days, about 2-15 days, about 2-14 days, about 2-10 days, about 2-7 days, about 2-5 days, about 2-3 days, about 5-30 days, about 5-28 days, about 5-21 days, about 5-20 days, about 5-15 days, about 5-14 days, about 5-10 days, about 5-7 days, about 7-30 days, about 7-28 days, about 7-21 days, about 7-20 days, about 7-15 days, about 7-14 days, about 7-10 days, about 14-30 days, about 14-28 days, about 14-21 days, about 14-20 days, about 14-15 days, about 15-30 days, about 15-28 days, about 15-21 days, about 15-20 days, about 20-30 days, about 20-28 days, about 20-21 days, about 21-30 days, about 21-28 days, and about 28-30 days as compared to administering the Clostridial toxin derivative in the absence of the chemical depolarizing agent.

In embodiments, the combined administration is effective to increase duration of the effect at least about 30 days, about 28 days, about 21 days, about 20 days, about 15 days, about 14 days, about 10 days, about 7 days, about 5 days, about 3 days, about 2 days, and about 1 days as compared to administering the Clostridial toxin derivative in the absence of the chemical depolarizing agent. In embodiments, the combined administration is effective to increase duration of the effect up to about 30 days, about 28 days, about 21 days, about 20 days, about 15 days, about 14 days, about 10 days, about 7 days, about 5 days, about 3 days, about 2 days, and about 1 days as compared to administering the Clostridial toxin derivative in the absence of the chemical depolarizing agent.

Without being limited as to theory, it is believed the chemical depolarizing agent increases neuronal uptake of the Clostridial toxin derivative by altering vesicular recycling. The chemical depolarizing agent increases vesicular recycling as a result of enhanced nerve terminal activity (exocytosis and endocytosis).

The chemical depolarizing agent may be selected from one or more of a potassium channel blocker, a calcium channel ionophore, a sodium channel ionophore, and potassium. In some embodiments, the potassium channel blocker is 4-aminopyridine (4-AP). 4-AP acts as a selective blocker of voltage-activated K+ channels, which leads to prolonged action potentials and increased neurotransmitter release in the neuromuscular junction. 4-AP has also been suggested to block voltage-dependent Na+ channels. In some embodiments, the potassium channel blocker is DAP. In some embodiments, the calcium channel ionophore is selected from ionmycin and calcimycin. In some embodiments, the sodium channel ionophore is selected from the group consisting of monensin and gramicidin. In some embodiments, the chemical depolarizing agent is potassium ion (K+). It will be appreciated that one or more chemical depolarizing agent may be administered to increase the therapeutic effectiveness of the Clostridial toxin derivative. Where two or more chemical depolarizing agents are administered, the agents may be administered in combination or separately (separate formulations as well as separate in time).

In one embodiment, the Clostridial toxin derivative is formulated in a solution comprising at least one chemical depolarizing agent. In some embodiments, where at least one of the chemical depolarizing agents is 4-AP, the Clostridial toxin derivative is formulated in about a 1 μM to 100 μM chemical depolarizing solution (e.g. 4-AP solution). In embodiments, the Clostridial toxin derivative is formulated in about a 10-100, 20-100, 30-100, 40-100, 50-100, 60-100, 70-100, 80-100, and 90-100 μM chemical depolarizing solution. In some embodiments, the Clostridial toxin derivative is formulated in a chemical depolarizing solution of at least about 1 μM to 100 μM. In some embodiments, the Clostridial toxin derivative is formulated in a chemical depolarizing solution of up to about 1 μM to 100 μM.

In one embodiment, BoNT/E is formulated in a solution comprising DAP. In some embodiments, BoNT/E is formulated in about a 1 μM to 100 μM DAP solution. In some embodiments, BoNT/E is formulated in about a 10-100, 20-100, 30-100, 40-100, 50-100, 60-100, 70-100, 80-100, and 90-100 μM DAP solution. In some embodiments, BoNT/E is formulated in a DAP solution of at least about 1 μM to 100 μM. In some embodiments, BoNT/E is formulated in a DAP solution of up to about 1 μM to 100 μM. In some embodiments, BoNT/E is formulated in a DAP solution of about 50 μM. In some embodiments, BoNT/E is about 10 pM or 100 pM in the formulated solution comprising, for example, 50 μM DAP.

In some embodiments, the Clostridial toxin derivative and/or the chemical depolarization agent are formulated in a sustained release composition. Sustained release compositions of botulinum neurotoxin have been described in U.S. Pat. Nos. 6,306,423, 6,312,708, 6,383,509, 6,506,399, 6,585,993, each of which are incorporated herein by reference. Sustained release compositions include those fabricated from bioerodible polymers (e.g. injectable microspheres), injectable implants, injectable gels or gelling systems, and topical microneedles. Bioerodible polymers can be used to form monolithic homogeneous or heterogeneous implants and microparticulates, membrane controlled implants or microparticulates, multistage delivery systems or a combination of these. Polymers of such implants can be natural or synthetic and may include polyesters, poly (ortho esters) or polyanhydrides, polysaccharides, polypeptides and other polymers known in the art. Specific, but not limiting polymers include poly-lactic acid (PLA), poly (lactide-co-glycolide) (PLGA), Poly-L-lactic acid (PLLA), polycaprolactone and poly (ortho acetate). Sustained release compositions may also be fabricated from other materials, such as non-erodible polymers, lipids or lipid-containing materials, or ceramics such as mesoporous silica.

In one embodiment, the chemical depolarization agent is incorporated into a sustained release formulation comprising the Clostridial toxin derivative. These formulations may be designed to release the Clostridial toxin derivative and the chemical depolarization agent together in a continuous release, or with one or both, released in a pulsatile manner. Although described as “sustained” release, the formulation may provide sustained, pulsatile or multiphasic release of the Clostridial toxin derivative and/or one or more chemical depolarization agents. The release profiles may be further designed to ensure simultaneous or sequential release of the Clostridial toxin derivative and the chemical depolarization agent. The chemical depolarization agent may also be administered in a sustained release formulation separately from the Clostridial toxin derivative. The Clostridial toxin derivative and one or more of the chemical depolarization agents may be administered by the same or different routes of administration.

A neurotoxin, such as a botulinum neurotoxin A and/or a tetrodotoxin, can be encapsulated in the polysaccharide polymer network itself or into large vessels within the network that enable local, sustained and controlled release of actives to the surrounding tissue of the injection site. Parameters such as hyaluronic acid concentration, degree of crosslinking, median particle size, and molecular weight of the raw material hyaluronic acid can be varied to permit altered release kinetics for the neurotoxin. The encapsulation can be carried out by simple mixing or by allowing the neurotoxin to mix with the polymer network at a highly hydrated state; followed by dehydration of the network to control the release kinetics (e.g. final swelling ratio of the polymer). In other embodiments, the release kinetics of the agents in the drug delivery system can be controlled via chemical degradation of the delivery system, or chemical or solvent activation. An external stimulus can also be used to control release of the agents. Examples of such stimuli include electric current, ultrasound, light, or thermal factors. The contracted network can be sized into particles, mixed with the dermal filler and delivered at the site of the injection. The slow re-hydration of the neurotoxin loaded polysaccharide particles can provide a sustained and controlled delivery of the neurotoxin. U.S. Pat. Nos. 7,691,381, 8,007,828, and 8,501,187 describe stabilized biodegradable neurotoxin implants and are each incorporated by reference herein.

In some embodiments, a kit is provided including the Clostridial toxin derivative and the one or more chemical depolarizing agent as a combined or separate formulations or compositions. The kit may include instructions for administering the composition or compositions.

### III. Methods of Treatment

In embodiments, methods of treating diseases, disorders, conditions, and the like, comprising the step or steps of administering a Clostridial toxin derivative and a chemical depolarizing agent in need thereof in amounts sufficient to produce improved patient function. In embodiments, administration of the Clostridial toxin derivative and the chemical depolarizing agent is effective to induce local, partial and/or complete muscle denervation in a subject. In embodiments, administration of the Clostridial toxin derivative and the chemical depolarizing agent is effective to induce local, partial and/or complete muscle paralysis in a subject. In embodiments, administration of the Clostridial toxin derivative and the chemical depolarizing agent is effective to treat diseases and/or conditions that are amenable to treatment by inducing local, partial and/or complete muscle denervation and/or paralysis. In certain embodiments, the diseases are of a neuromuscular nature, such as, for example, those diseases that affect muscles and nerve control thereof, such as, for example, overactive bladder, and the like. Certain embodiments relate to the treatment of pain, such as, for example, treatment of headache pain, or back pain, or muscle pain, or the like. In certain embodiments, methods of the invention encompass the treatment of psychological disorders, including, for example, depression, anxiety, and the like. In certain embodiments, methods of the invention encompass the treatment of skin disorders, including, for example, a wart, bunion, callus, corn, ulcer, neuroma, hammertoe, dermatofibroma, keloid, mole (such as a typical mole or dysplastic nevi), granuloma (such as a pyogenic granuloma), a keratose (such as a seborrheic keratose), and skin pigment disorders (melanin related afflictions) including melasma, pigment loss after skin damage, vitiligo, freckles and moles (nevi). Skin disorder can be noncancerous skin pigment disorders such as dermatofibromas, dermoid cyst, freckles, keloids, keratoacanthomas, lipomasmoles (nevi), atypical moles (dysplastic nevi), pyogenic granulomas, seborrheic keratoses, actinic keratosis, skin tags, pigment disorders: albinism, melasma, pigment loss after skin damage and vitiligo. Skin disorder can also be melanin disregulation related skin cancers include melanoma, basal cell carcinoma and squamous cell carcinoma. In certain embodiments, methods of the invention encompass the treatment of wounds and scars.

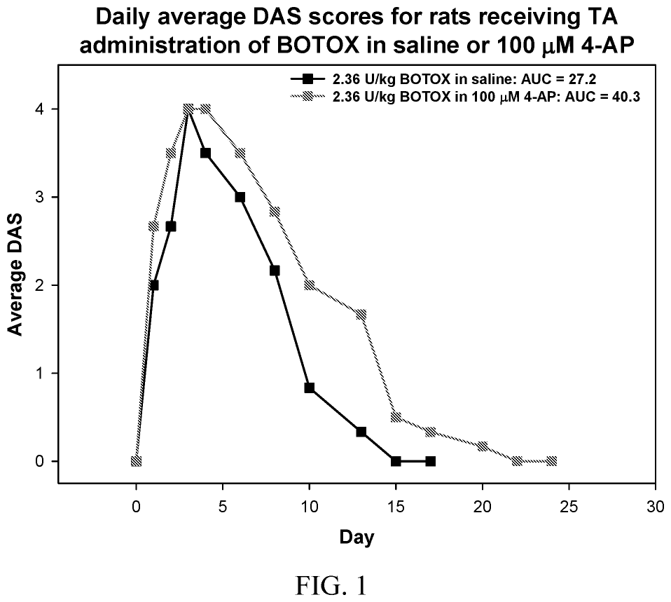

As described in Example 1, i.m. injection of BOTOX® (2.36 U/kg) formulated in saline resulted in an average DAS score of 4.0 at 4 days post-injection in rats. The DAS score for the BOTOX® in saline recovered to a baseline level (0) after about two weeks post-injection. The average DAS score for the rats injected with BOTOX® in 100 μM 4-AP was 4.0 at 4 days post-injection, which recovered to baseline level (0) only at about three weeks post-injection. Thus, injection of BOTOX® with a chemical depolarization agent had a longer duration of muscle paralysis for the equivalent dose of BOTOX® without the chemical depolarization agent. The DAS score for the BOTOX® formulated with the chemical depolarizing agent remained higher than the DAS score for BOTOX® formulated in saline for over two weeks. Area under the curve (AUC) analysis of the graph of FIG. 1 demonstrated that BOTOX® formulated a 4-AP solution resulted in a larger AUC than BOTOX® formulated in saline showing a greater effect on paralysis for injection of BOTOX® formulated with a chemical depolarization agent.

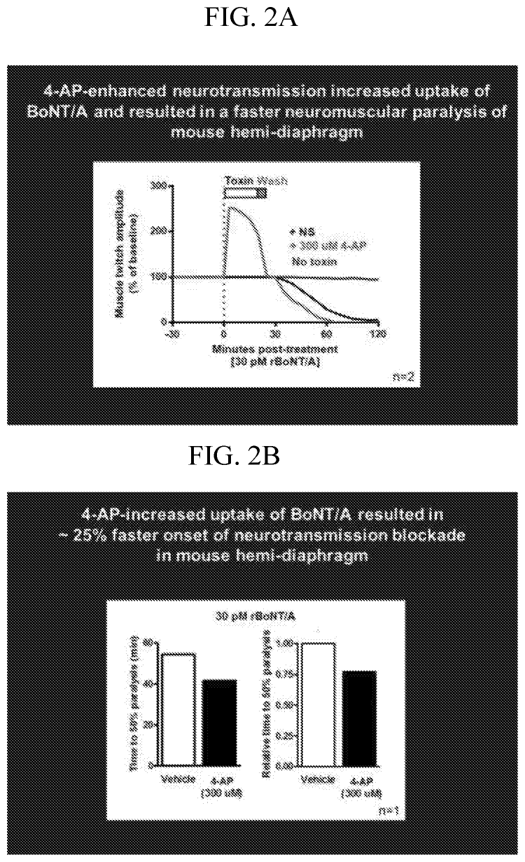

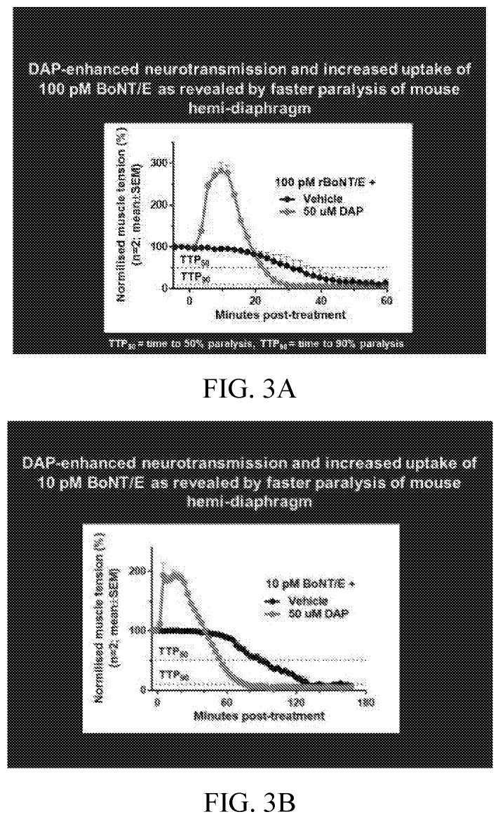

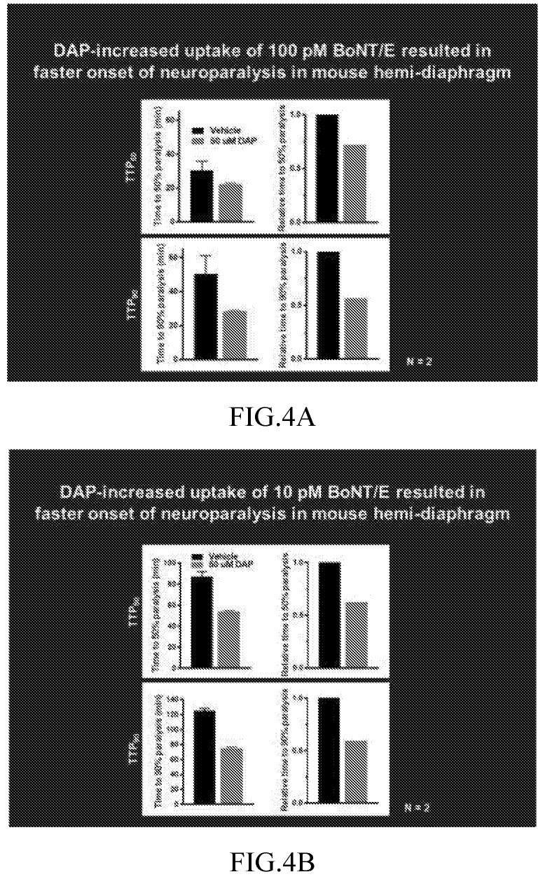

In another study using the mouse phrenic nerve hemidiaphragm, administration of 300 μM of 4-AP before 30 pM BoNT/A raised the initial amplitude of muscle tension by approximately 2.5-fold, while the onset of paralysis progressed faster than with 30 pM BoNT/A in the absence of 4-AP, as seen in the data presented in FIGS. 2A-2B. In another study, administration of 50 μM DAP in combination with 100 or 10 pM BoNT/E led to an elevated muscle tension response, which was followed by a faster muscle denervation as compared to administration of BoNT/E alone, as seen in the data shown in FIGS. 3A-3B. From this data, it can be extrapolated that a 6-times higher concentration of toxin would be required to induce an equivalent reduction in time for 90% paralysis that was caused by DAP, thus, potentially allowing use of less toxin to get the same effect. Moreover, DAP increased uptake of BoNT/E, resulting in faster onset of neuroparalysis in the mouse phrenic nerve hemi-diaphragm, as seen in the data presented in FIGS. 4A-4B.

These studies demonstrate that formulating a Clostridial toxin derivative with one or more chemical depolarizing agents and a combination therapy where the Clostridial toxin derivative is administered within a short period of time of one or more chemical depolarization agents extends the muscle weakening effect of the Clostridial toxin derivative, accelerates the onset of a Clostridial toxin derivative intoxication, and/or reduces the therapeutically effective amount of the Clostridial toxin derivative as compared to that of the Clostridial toxin derivative alone by about 25-600%. For example, DAP accelerates the onset of BoNT/E intoxication in the mouse phrenic nerve hemi-diaphragm in the mouse phrenic nerve hemi-diaphragm. Further for example, DAP can reduce the therapeutically effective amount of BoNT/E as compared to that of BoNT/E administered alone by about 25-600%.

Compositions, formulations and methods can be useful for the treatment, reduction of symptoms, and/or prevention of, for example, achalasia, anal fissure, anismus, blepharospasm, cerebral palsy, cervical dystonia, cervicogenic headache, hemifacial spasm, dyshidrotic eczema, dysphagia, dysphonia, esophageal dysmotility, esophageal muscular ring, esotropia (infantile), eyelift, facial myokemia, gait disturbances (idiopathic toe-walking), generalized dystonia, hemifacial spasm, hyperfunctional facial lines (glabellar, forehead, crows' feet, down-turned angles of the mouth), hyperhidrosis, incontinence (idiopathic or neurogenic), medication overuse headache, migraine headache, myoclonus, muscle mass or activity reduction, involving, for example, the masseter or the like, myofascial pain syndrome, obstructive urinary symptoms, pancreas divisum pancreatitis, Parkinson's disease, puborectalis syndrome, reduction of surgical scar tension, salivary hypersecretion, sialocele, sixth nerve palsy, spasticity, speech/voice disorders, strabismus, surgery adjunct (ophthalmic), tardive dyskinesia, temporomandibular joint disorders, tension headache, thoracic outlet syndrome, torsion dystonia, torticolis, Tourette's syndrome, tremor, whiplash-associated neck pain, pain, itching, inflammation, allergy, cancer and benign tumors, fever, obesity, infectious diseases, viral and bacterial, hypertension, cardiac arrhythmias, vasospasm, atherosclerosis, endothelial hyperplasia, venous thrombosis, varicose veins, apthous stomatitis, hypersalivation, temporomandibular joint syndrome, hyperhidrosis, bromhidrosis, acne, rosacea, hyperpigmentation, hypertrophic scars, keloids, calluses and corns, skin wrinkling, excessive sebum production, psoriasis, dermatitis, allergic rhinitis, nasal congestion, post nasal drip, sneezing, ear wax, serous and suppurative otitis media, tonsil and adenoid hypertrophy, tinnitus, dizziness, vertigo, hoarseness, cough, sleep apnea, snoring, glaucoma, conjunctivitis, uveitis, strabismus, Grave's disease, excessive hair growth, hair loss, asthma, bronchitis, emphysema, mucus production, pleuritis, coagulation disorders, myeloproliferative disorders, disorders involving eosinophils, neutrophils, macrophages and lymphocytes, immune tolerance and transplantation, autoimmune disorders, dysphagia, acid reflux, hiatal hernia, gastritis and hyperacidity, diarrhea and constipation, hemorrhoids, urinary incontinence, prostatic hypertrophy, erectile dysfunction, priapism and Peyronie's disease, epididymitis, contraception, menstrual cramps, preventing premature delivery, endometriosis and fibroids, arthritis, osteoarthritis, rheumatoid, bursitis, tendonitis, tenosynovitis, fibromyalgia, seizure disorders, spasticity, headache, and neuralgias.

The Clostridial toxin derivative and the one or more chemical depolarization agents may be administered at the same time or within a specified time period. In embodiments, the Clostridial toxin derivative and the one or more chemical depolarization agents may be administered in the same formulation. In some embodiments, a Clostridial toxin derivative formulation may be mixed or combined with the one or more chemical depolarization agents. Where the Clostridial toxin derivative formulation is vacuum dried or lyophilized or incorporated into a dissolving matrix or microneedle patch, the Clostridial toxin derivative formulation may be reconstituted in a vehicle containing the one or more chemical depolarization agents. Alternatively, the one or more chemical depolarization agents may be included in the vacuum dried or lyophilized formulation or dissolving matrix formulation. In some embodiments, the Clostridial toxin derivative and the chemical depolarization agent are administered in separate formulations. In some embodiments, the Clostridial toxin derivative and the chemical depolarization agent formulation are administered at the same or substantially the same time. In embodiments, the Clostridial toxin derivative is administered (e.g. injected) prior to or following administration of the one or more chemical depolarization agents.

In embodiments, the Clostridial toxin derivative is administered at least about 1 minute to about 24 hours prior to or following administration of the chemical depolarization agent. In embodiments, the Clostridial toxin derivative and the chemical depolarizing agent are administered within about 1 minute to about 18hours, about 1 minute to about 12 hours, about 1 minute to about 10 hours, about 1 minute to about 8 hours, about 1 minute to about 6 hours, about 1 minute to about 5 hours, about 1 minute to about 4 hours, about 1 minute to about 3 hours, about 1 minute to about 2 hours, about 1 minute to about 1 hour, about 1-45 minutes, about 1-30 minutes, about 1-20 minutes, about 1-15 minutes, about 1-10 minutes, about 1-5 minutes, about 1-3 minutes, about 1-2 minutes, about 2 minutes to about 24 hours, about 2 minutes to about 18hours, about 2 minutes to about 12 hours, about 2 minutes to about 10 hours, about 2 minutes to about 8 hours, about 2 minutes to about 6 hours, about 2 minutes to about 5 hours, about 2 minutes to about 4 hours, about 2 minutes to about 3 hours, about 2 minutes to about 2 hours, about 2 minutes to about 1 hour, about 2-45 minutes, about 2-30 minutes, about 2-20 minutes, about 2-15 minutes, about 2-10 minutes, about 2-5 minutes, about 2-3 minutes, about 3 minutes to about 24 hours, about 3 minutes to about 18hours, about 3 minutes to about 12 hours, about 3 minutes to about 10 hours, about 3 minutes to about 8 hours, about 3 minutes to about 6 hours, about 3 minutes to about 5 hours, about 3 minutes to about 4 hours, about 3 minutes to about 3 hours, about 3 minutes to about 2 hours, about 3 minutes to about 1 hour, about 3-45 minutes, about 3-30 minutes, about 3-20 minutes, about 3-15 minutes, about 3-10 minutes, about 3-5 minutes, about 5 minutes to about 24 hours, about 5 minutes to about 18 hours, about 5 minutes to about 12 hours, about 5 minutes to about 10 hours, about 5 minutes to about 8 hours, about 5 minutes to about 6 hours, about 5 minutes to about 5 hours, about 5 minutes to about 4 hours, about 5 minutes to about 3 hours, about 5 minutes to about 2 hours, about 5 minutes to about 1 hour, about 5-45 minutes, about 5-30 minutes, about 5-20 minutes, about 5-15 minutes, about 5-10 minutes, about 10 minutes to about 24 hours, about 10 minutes to about 18hours, about 10 minutes to about 12 hours, about 10 minutes to about 10 hours, about 10 minutes to about 8 hours, about 10 minutes to about 6 hours, about 10 minutes to about 5 hours, about 10 minutes to about 4 hours, about 10 minutes to about 3 hours, about 10 minutes to about 2 hours, about 10 minutes to about 1 hour, about 10-45 minutes, about 10-30 minutes, about 10-20 minutes, about 10-15 minutes, about 15 minutes to about 24 hours, about 15 minutes to about 18hours, about 15 minutes to about 12 hours, about 15 minutes to about 10 hours, about 15 minutes to about 8 hours, about 15 minutes to about 6 hours, about 15 minutes to about 5 hours, about 15 minutes to about 4 hours, about 15 minutes to about 3 hours, about 15 minutes to about 2 hours, about 15 minutes to about 1 hour, about 15-45 minutes, about 15-30 minutes, about 15-20 minutes, about 20 minutes to about 24 hours, about 20 minutes to about 18hours, about 20 minutes to about 12 hours, about 20 minutes to about 10 hours, about 20 minutes to about 8 hours, about 20 minutes to about 6 hours, about 20 minutes to about 5 hours, about 20 minutes to about 4 hours, about 20 minutes to about 3 hours, about 20 minutes to about 2 hours, about 20 minutes to about 1 hour, about 20-45 minutes, about 20-30 minutes, about 30 minutes to about 24 hours, about 30 minutes to about 18hours, about 30 minutes to about 12 hours, about 30 minutes to about 10 hours, about 30 minutes to about 8 hours, about 30 minutes to about 6 hours, about 30 minutes to about 5 hours, about 30 minutes to about 4 hours, about 30 minutes to about 3 hours, about 30 minutes to about 2 hours, about 30 minutes to about 1 hour, about 30-45 minutes, about 45 minutes to about 24 hours, about 45 minutes to about 18hours, about 45 minutes to about 12 hours, about 45 minutes to about 10 hours, about 45 minutes to about 8 hours, about 45 minutes to about 6 hours, about 45 minutes to about 5 hours, about 45 minutes to about 4 hours, about 45 minutes to about 3 hours, about 45 minutes to about 2 hours, about 45 minutes to about 1 hour, about 1-24 hours, about 1-18hours, about 1-12 hours, about 1-10 hours, about 1-8 hours, about 1-6 hours, about 1-5 hours, about 1-4 hours, about 1-3 hours, about 1-2 hours, about 2-24 hours, about 2-18hours, about 2-12 hours, about 2-10 hours, about 2-8 hours, about 2-6 hours, about 2-5 hours, about 2-4 hours, about 2-3 hours, about 3-24 hours, about 3-18 hours, about 3-12 hours, about 3-10 hours, about 3-8 hours, about 3-6 hours, about 3-5 hours, about 3-4 hours, about 4-24 hours, about 4-18 hours, about 4-12 hours, about 4-10 hours, about 4-8 hours, about 4-6 hours, about 4-5 hours, about 5-24 hours, about 5-18hours, about 5-12 hours, about 5-10 hours, about 5-8 hours, about 5-6 hours, about 6-24 hours, about 6-18 hours, about 6-12 hours, about 6-10 hours, about 6-8 hours, about 8-24 hours, about 8-18 hours, about 8-12 hours, about 8-10 hours, about 10-24 hours, about 10-18 hours, about 10-12 hours, about 12-24 hours, about 12-18 hours, or about 18-24 hours.

In embodiments, the Clostridial toxin derivative and the chemical depolarizing agent are administered less than 1 minute, 2 minutes, 3 minutes, 4 minutes, 5 minutes, 10 minutes, 15 minutes, 20 minutes, 30 minutes, 45 minutes, 1 hour, 1.5 hours, 2 hours, 3 hours, 4 hours, 5 hours, 6 hours, 8 hours, 10 hours, 12 hours, 18hours, or 24 hours apart. In embodiments, the Clostridial toxin derivative and the chemical depolarizing agent are administered greater than or equal to than 1 minute, 2 minutes, 3 minutes, 4 minutes, 5 minutes, 10 minutes, 15 minutes, 20 minutes, 30 minutes, 45 minutes, 1 hour, 1.5 hours, 2 hours, 3 hours, 4 hours, 5 hours, 6 hours, 8 hours, 10 hours, 12 hours, 18hours, or 24 hours apart.

The Clostridial toxin derivative and the chemical depolarizing agent may be administered by any suitable means as known in the art. It will be appreciated that the means of administration may be different for the Clostridial toxin derivative and the chemical depolarizing agent. In some embodiments, at least one of the Clostridial toxin derivative and the chemical depolarizing agent is locally administered. In some embodiments, at least one of the Clostridial toxin derivative and the chemical depolarizing agent is administered by intramuscular, intradermal, subdermal injection, or topical administration. It will further be appreciated that the Clostridial toxin derivative and the one or more chemical depolarization agent may be administered at or near the same site (e.g. injection) or at different sites/routes of administration.

### Neuromuscular Conditions

In an embodiment, the neuromuscular disease is hyperhidrosis. A subject suffering from hyperhidrosis, for example, receives about 59 U per axilla, or about 58 U per axilla, or about 57 U per axilla, or about 56 U per axilla, or about 55 U per axilla, or about 54 U per axilla, or about 53 U per axilla, or about 52 U per axilla, or about 51 U per axilla, or about 50 U per axilla, or about 49 U per axilla, or about 48 U per axilla, or about 47 U per axilla, or about 46 U per axilla, or about 45 U per axilla, or about 44 U per axilla, or about 43 U per axilla, or about 42 U per axilla, or about 41 U per axilla, or about 40 U per axilla, or about 39 U per axilla, or about 38 U per axilla, or about 37 U per axilla, or about 36 U per axilla, or less, per treatment of a Clostridial derivative in a pharmaceutical formulation. In an embodiment, a formulation comprising 50 U total are injected intradermally into 10-15 sites spaced approximately 1-2 cm apart.

In an embodiment, the neuromuscular disease is hemifacial spasm. A subject suffering from hemifacial spasm, for example receives a formulation comprising between about 1.5 to 15 U per treatment of a Clostridial derivative in a pharmaceutical formulation. In a further example, the subject receives between about 1.5 to 3 U, 1.5 to 5 U, 1.5 to 7 U, 1.5 to 10 U, 1.5 to 12 U, 1.5 to 15 U, 5 to 10 U, 5 to 15 U, or 10 to 15 U of a Clostridial derivative per treatment administered to a patient with hemifacial spasm. In a still further example, the subject receives about 1.5 U, about 2 U, about 2.5 U, about 3 U, about 3.5 U, about 4 U, about 4.5 U about 5 U, about 5.5 U, about 6 U, about 6.5 U, about 7 U, about 7.5 U, about 8 U, about 8.5 U, about 9 U, about 9.5 U, about 10 U, about 10.5 U, about 11 U, about 11.5 U, about 12 U, about 12.5 U, about 13 U, about 13.5 U, about 14 U, about 14.5 U, or about 15 U of a Clostridial derivative per treatment are administered to a patient with hemifacial spasm. Dosages greater than 15 U of a Clostridial derivative per treatment may also be administered to patients with hemifacial spasm to achieve a therapeutic response. A treatment session can comprise multiple treatments.

In an embodiment, the neuromuscular disease is cervical dystonia. A subject suffering from cervical dystonia, for example, receives between about 15 to 300 U per treatment of a Clostridial derivative in a pharmaceutical formulation. In a further example, the subject receives between about 35 to 250 U, 65 to 200 U, 85 to 175 U, 105 to 160 U, or 125 to 145 U are administered to a patient with cervical dystonia. In an embodiment, dosages to the sternocleidomastoid muscle are limited to 100 U or less. Dosages greater than 300 U per treatment may also be administered to patients with cervical dystonia to achieve a therapeutic response. A treatment session can comprise multiple treatments.

In an embodiment, the neuromuscular disease is blepharospasm. A subject suffering from blepharospasm, for example, receives between about 1.25 to 2.5 U of a Clostridial derivative in a pharmaceutical formulation injected into the medial and lateral pretarsal orbicularis oculi of the upper lid and into the lateral pretarsal orbicularis oculi of the lower lid. In a further example, the subject receives about 1.5 U, about 1.6 U, about 1.7 U, about 1.8 U, about 1.9 U, about 2.0 U, about 2.1 U, about 2.2 U, about 2.3 U, about 2.4 U, about 2.5 U, or more of the Clostridial derivative, per injection site. A treatment session can comprise multiple treatments.

In an embodiment, the neuromuscular disease is strabismus. A subject suffering from strabismus, for example, receives between about 1.25 to 2.5 U per injection site of a Clostridial derivative in a pharmaceutical formulation. In a further example, the subject receives about 1.5 U, about 1.6 U, about 1.7 U, about 1.8 U, about 1.9 U, about 2.0 U, about 2.1 U, about 2.2 U, about 2.3 U, about 2.4 U, about 2.5 U, or more, of a Clostridial derivative per injection site to achieve a therapeutic response. In embodiments, lower doses are used for treatment of small deviations. In embodiments, vertical muscles and horizontal strabismus of less than 20 prism diameters can be treated with 1.25 to 2.5 U per injection site. A treatment session can comprise multiple treatments.

In an embodiment, the neuromuscular disease is muscle spasticity. A subject suffering from muscle spasticity, for example, receives between about 20 to 200 U per treatment of a Clostridial derivative in a pharmaceutical formulation. In a further example, the subject receives between about 20 to 30 U, 20 to 40 U, 20 to 60 U, 20 to 80 U, 20 to 100 U, 20 to 125 U, 20 to 150 U, or 20 to 175 U of a Clostridial derivative per treatment are administered to a patient with muscle spasticity. In a still further example, the subject receives about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 155 U, about 160 U, about 165 U, about 170 U, about 175 U, about 180 U, about 185 U, about 190 U, about 195 U, or about 200 U per treatment are administered to a patient with muscle spasticity. In an embodiment, the biceps brachii can be injected with between 100 U and 200 U divided into 4 injection sites. In an embodiment, the flexor carpi radialis can be injected with between 12.5 U and 50 U in 1 injection site. In an embodiment, the flexor carpi ulnaris can be injected with between 12.5 U and 50 U in 1 injection site. In an embodiment, the flexor digitorum profundus can be injected with between 30 U and 50 U in one injection site. In an embodiment, the flexor digitorum sublimis can be injected with between 30 U and 50 in a single injection site. Dosages greater than 200 U per treatment may also be administered to patients with muscle spasticity to achieve a therapeutic response. A treatment session can comprise multiple treatments.

### Pain

In another embodiment, methods for treating pain are provided that comprise the step of administering a pharmaceutical formulation comprising a Clostridial derivative and a chemical depolarizing agent to a subject in need thereof in an amount sufficient to reduce pain. In another embodiment, the patient suffers from myofascial pain, migraine headache pain, tension headache pain, neuropathic pain, facial pain, lower-back pain, sinus-headache pain, pain associated with temporomandibular joint disease, pain associated with spasticity or cervical dystonia, post-surgical wound pain, or neuralgia. A treatment session can comprise multiple treatments.

In an embodiment, the patient suffers from facial pain. A subject suffering from facial pain, for example, receives between about 4 to 40 U of a Clostridial derivative per treatment of a pharmaceutical formulation. In a further example, the subject receives between about 4 to 10 U, 4 to 15 U, 4 to 20 U, 4 to 25 U, 4 to 30 U, 4 to 35 U, 7 to 15 U, 7 to 20 U, 7 to 25 U, 7 to 30 U, 7 to 35 U, or 7 to 40 U per treatment administered to a patient suffering from facial pain. In a still further example, the subject receives about 4 U, about 5 U, about 7.5 U, about 10 U, about 12.5 U, about 15 U, about 17.5 U, about 20.0 U, about 22.5 U, about 25.0 U, about 27.5 U, about 30.0 U, about 32.5 U, about 35 U, about 37.5 U, or about 40 U per treatment are administered to a patient with facial pain. Dosages greater than 40 U per treatment may also be administered to patients with facial pain to achieve a therapeutic response. A treatment session can comprise multiple treatments.

In an embodiment, the patient suffers from myofascial pain. A subject suffering from myofascial pain, for example, receives between about 5 to 100 U of a Clostridial derivative per treatment of a pharmaceutical formulation. In a further example, the subject receives between about 5 to 10 U, 5 to 20 U, 5 to 30 U, 5 to 40 Units, 5 to 50 Units, 5 to 60 Units, 5 to 70 Units, 5 to 80 Units, 5 to 90 U, 10 to 20 U, 10 to 30 U, 10 to 50 U, or 10 to 60 U, or 10 to 70 U, or 10 to 80 U, 10 to 90 U, or 10 to 100 U of a Clostridial derivative per treatment are administered to a patient suffering from myofascial pain. In a further example, the subject receives about 5 U, about 10 U, about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, or about 100 U of a Clostridial derivative per treatment are administered to a patient with myofascial pain. Dosages greater than 100 U per treatment may also be administered to patients with myofascial pain to achieve a therapeutic response. A treatment session can comprise multiple treatments.

In an embodiment, the subject suffers from lower-back pain. A subject suffering from lower-back pain, for example, receives between about 15 to 150 U of a Clostridial derivative per treatment of a pharmaceutical formulation. In a further example, the subject receives between about 15 to 30 U, 15 to 50 U, 15 to 75 U, 15 to 100 U, 15 to 125 U, 15 to 150 U, 20 to 100 U, 20 to 150 U, or 100 to 150 U of a Clostridial derivative per treatment are administered to a patient with lower-back pain. In a still further example, the subject receives about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, or about 150 U of a Clostridial derivative per treatment are administered to a patient with lower-back pain. Dosages greater than 150 U per treatment may also be administered to patients with lower-back pain to achieve a therapeutic response. A treatment session can comprise multiple treatments.

In an embodiment, the patient suffers from migraine headache pain, including wherein the patient suffers from migraine headaches of 4 hours or more 15 or more days per month. A subject suffering from migraine-headache pain, for example, receives between about 0.5 to 200 U of a Clostridial derivative per treatment of a pharmaceutical formulation. In a further example, the subject receives between about 5 to 190 U, 15 to 180 U, 25 to 170 U, 35 to 160 U, 45 to 150 U, 55 to 140 U, 65 to 130 U, 75 to 120 U, 85 to 110 U, or 95 to 105 U of a Clostridial derivative per treatment are administered to a patient suffering from migraine headache pain. A treatment session can comprise multiple treatments.

For example, about 0.5 U, about 1.0 U, about 1.5 U, about 2.0 U, about 2.5 U, about 3.0 U, about 3.5 U, about 4.0 U, about 4.5 U, about 5.0 U, about 5.5 U, about 6.0 U, about 6.5 U, about 7.0 U, about 7.5 U, about 8.0 U, about 8.5 U, about 9.0 U, about 9.5 U, about 10.0 U, about 12 U, about 15 U, about 17 U, about 20 U, about 22 U, about 25 U, about 27 U, about 30 U, about 32 U, about 35 U, about 37 U, about 40 U, about 42 U, about 45 U, about 47 U, or about 50 U of a Clostridial derivative per treatment site are administered to a patient with migraine-headache pain. A patient can be treated at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, or more, or the like. In an embodiment, a patient suffering from migraine is injected 31 times with 5 U per 0.1 mL injection, across the corrugator (2 injections of 5 U each), procerus (1 injection of 5 U), frontalis (4 injections of 5 U each), temporalis (8 injections of 5 U each), occipitalis (6 injections of 5 U each), cervical paraspinal (4 injections of 5 U each), and trapezius (6 injections of 5 U each) muscles. With the exception of the procerus muscle which can be injected at the midline, all muscles can, in certain embodiments, be injected bilaterally with half of the injection sites to the left and half to the right side of the head and neck. Dosages greater than 200 U per treatment may also be administered to patients with migraine-headache pain to achieve a therapeutic response. A treatment session can comprise multiple treatments.

In an embodiment, the patient suffers from sinus-headache pain. A subject suffering from sinus-headache pain, for example, receives between about 4 to 40 U of a Clostridial derivative per treatment of a pharmaceutical formulation. In a further example, the subject receives between about 4 to 10 U, 4 to 15 U, 4 to 20 U, 4 to 25 U, 4 to 30 U, 4 to 35 U, 7 to 15 U, 7 to 20 U, 7 to 25 U, 7 to 30 U, 7 to 35 U, or 7 to 40 U of a Clostridial derivative per treatment that is administered to a patient suffering from sinus-headache pain. In a still further example, the subject receives about 4 U, about 5 U, about 7.5 U, about 10 U, about 12.5 U, about 15 U, about 17.5 U, about 20.0 U, about 22.5 U, about 25.0 U, about 27.5 U, about 30.0 U, about 32.5 U, about 35 U, about 37.5 U, or about 40 U of a Clostridial derivative per treatment are administered to a patient with sinus-headache pain. Dosages greater than 40 U per treatment may also be administered to patients with sinus headache-pain to achieve a therapeutic response. A treatment session can comprise multiple treatments.

In an embodiment, the patient suffers from tension-headache pain. A subject suffering from tension-headache pain, for example, receives between about 5 to 50 U of a Clostridial derivative per treatment of a pharmaceutical formulation. In a further example, the subject receives between about 5 to 10 U, 5 to 15 U, 5 to 20 U, 5 to 25 U, 5 to 30 U, 5 to 35 U, 5 to 40 U, 5 to 45 U, 10 to 20 U, 10 to 25 U, 10 to 30 U, 10 to 35 U, 10 to 40 U, or 10 to 45 U of a Clostridial derivative per treatment that is administered to a patient with tension-headache pain. In a still further example, the subject receives about 5 U, about 10 U, about 20 U, about 25 U, about 30 U, about 35 U, about 40 U, about 45 U, or about 50 U of a Clostridial derivative per treatment that is administered to a patient with tension-headache pain. In an embodiment, a patient suffering from tension headache is injected 31 times with 5 U per 0.1 mL injection, across the corrugator (2 injections of 5 U each), procerus (1 injection of 5 U), frontalis (4 injections of 5 U each), temporalis (8 injections of 5 U each), occipitalis (6 injections of 5 U each), cervical paraspinal (4 injections of 5 U each), and trapezius (6 injections of 5 U each) muscles. With the exception of the procerus muscle which can be injected at the midline, all muscles can, in certain embodiments, be injected bilaterally with half of the injection sites to the left and half to the right side of the head and neck. Dosages greater than 200 U per treatment may also be administered to patients with tension headache pain to achieve a therapeutic response. A treatment session can comprise multiple treatments.

In an embodiment, the patient suffers from sinus headache pain or facial pain associated with acute or recurrent chronic sinusitis. For example, the pharmaceutical formulation can be administered to the nasal mucosa or to the subcutaneous structures overlying the sinuses, wherein the administration of the formulation reduces the headache and/or facial pain associated with acute recurrent or chronic sinusitis. In further embodiments, any of the pharmaceutical formulations described herein can be administered to the nasal mucosa or to the subcutaneous structures overlying the sinuses, such as over one or more of the sinuses selected from the group consisting of: ethmoid; maxillary; mastoid; frontal; and sphenoid. In another embodiment, subcutaneous structures overlying the sinuses lie within one or more of the areas selected from the group consisting of: forehead; malar; temporal; post auricular; and lip. In embodiments, multiple injections of a formulation comprising 5 U of Clostridial derivative each are administered to treat the sinus headache pain or facial pain associated with acute or recurrent chronic sinusitis.

In another embodiment, a patient suffering from sinus headache pain or facial pain associated with acute or recurrent chronic sinusitis is treated by administering any of the pharmaceutical formulations to an afflicted area of the patient. In a further embodiment, the pharmaceutical formulations disclosed herein are administered to the projections of a trigeminal nerve innervating a sinus.

Patients suffering from sinus headache pain or facial pain associated with acute or recurrent chronic sinusitis often exhibit symptoms including rhinitis, sinus hypersecretion and/or purulent nasal discharge. In one embodiment, patients treated with the pharmaceutical formulations exhibit symptoms of sinus hypersecretion and purulent nasal discharge.

In other embodiments, methods for treating a patient suffering from sinus headache pain or facial pain associated with acute or recurrent chronic sinusitis, wherein the subject suffers from neuralgia are provided. In certain embodiments the neuralgia is trigeminal neuralgia. In another embodiment, the neuralgia is associated with compressive forces on a sensory nerve; associated with intrinsic nerve damage, demyelinating disease, or a genetic disorder; associated with a metabolic disorder; associated with central neurologic vascular disease; or associated with trauma. In another embodiment of the present invention, the pain is associated with dental extraction or reconstruction.

### Urological Disorders

In an embodiment, methods for treating a patient suffering from overactive bladder (OAB), such as, for example, that due to a neurologic condition (NOAB), or idiopathic OAB (IOAB) are provided. For example, pharmaceutical formulations described herein can be administered to the bladder or its vicinity, e.g. the detrusor, wherein the administration of the formulation reduces the urge incontinence associated with overactive bladder. In certain embodiments, the dosage can be, for example, 200 U, or more, or less, or the like. For example, the dosage can be about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 160 U, about 170 U, about 180 U, about 190 U, about 200 U, about 210 U, about 220, about 230 U, about 240 U, or more, or the like, per treatment. A patient may be injected at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, 33 sites, 34 sites, 35 sites, 36 sites, 37 sites, 38 sites, or more, or the like. In an embodiment, patients suffering from OAB are treated with 30 1 mL injections of approximately 6.7 U per injection into the detrusor muscle.

In an embodiment, methods for treating a patient suffering from neurogenic detrusor overactivity (NDO), such as that due to a neurologic condition are provided herein. For example, pharmaceutical formulations as described herein can be administered to the bladder or its vicinity, e.g. the detrusor, wherein the administration of the formulation reduces the urge incontinence associated with overactive bladder. In certain embodiments, the dosage can be, for example, 200 U, or more, or less, or the like. For example, the dosage can be about 15 U, about 20 U, about 25 U, about 30 U, about 35 U, about 40 U, about 45 U, about 50 U, about 55 U, about 60 U, about 65 U, about 70 U, about 75 U, about 80 U, about 85 U, about 90 U, about 95 U, about 100 U, about 105 U, about 110 U, about 115 U, about 120 U, about 125 U, about 130 U, about 135 U, about 140 U, about 145 U, about 150 U, about 160 U, about 170 U, about 180 U, about 190 U, about 200 U, about 210 U, about 220, about 230 U, about 240 U, or more, or the like, per treatment. A patient can be injected at multiple sites, such as, for example, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites, 18 sites, 19 sites, 20 sites, 21 sites, 22 sites, 23 sites, 24 sites, 25 sites, 26 sites, 27 sites, 28 sites, 29 sites, 30 sites, 31 sites, 32 sites, or more, or the like. In an embodiment, patients suffering from NDO are treated with 30 1 mL injections of approximately 6.7 U per injection into the detrusor muscle.

### Cosmetic Uses

In another embodiment, methods for cosmetically modifying soft-tissue features comprise the step of administering a pharmaceutical formulation comprising a Clostridial derivative and a chemical depolarizing agent to a subject in need thereof in an amount sufficient to modify said features are provided. In a further embodiment, the pharmaceutical formulation is administered via transcutaneous or transmucosal injection either at a single focus or multiple foci.

In embodiments, pharmaceutical formulations described herein are administered to the face or neck of the subject. In a further embodiment, the pharmaceutical formulations described herein are administered to the subject in an amount sufficient to reduce rhytides. For example, the formulation can be administered between eyebrows of the subject in an amount sufficient to reduce vertical lines between the eyebrows and on a bridge of a nose. The pharmaceutical formulations can also be administered near either one or both eyes of the subject in an amount sufficient to reduce lines at corners of the eyes. In an embodiment, compositions of the invention can be injected locally to smooth skin. In another embodiment, the pharmaceutical formulations of the present invention can also be administered to a forehead of the subject in an amount sufficient to reduce horizontal lines on said forehead. In yet another embodiment of the present invention the pharmaceutical formulation is administered to the neck of the subject in an amount sufficient to reduce muscle bands in the neck. In an embodiment, a pharmaceutical composition is applied to the masseter muscle to relax the muscle and/or decrease masseter mass.

In a further embodiment, the patient suffers from facial wrinkles. A subject suffering from facial wrinkles, for example, can receive between about 1 to 100 U per treatment of a pharmaceutical formulation of the present invention. In a further example, the subject receives between about 1 to 10 U, 1 to 20 U, 1 to 30 U, 1 to 40 U, 1 to 50 U, 1 to 60 U, 1 to 70 U, 1 to 80 U, 1 to 90 U, 5 to 20 U, 5 to 30 U, 5 to 40 U, 5 to 50 U, 5 to 60 U, 5 to 70 U, 5 to 80 U, 5 to 90 U, or 5 to 100 U per treatment are administered to a patient with an inflammatory disorder. In a still further example, the subject receives about 1 U, about 10 U, about 20 U, about 30 U, about 40 U, about 50 U, about 60 U, about 70 U, about 80 U, about 90 U, or about 100 U per treatment are administered to a patient. Dosages greater than 100 U per treatment may also be administered to patients suffering from inflammation or an inflammatory disorder to achieve a therapeutic response.

### Inflammation

In another embodiment, methods for treating inflammation comprise the step of administering a pharmaceutical formulation comprising a Clostridial derivative and a chemical depolarizing agent to a subject in need thereof in an amount sufficient to reduce inflammation are provided. In certain embodiments, pharmaceutical formulations described herein are administered to a patient without producing muscle weakness. In an embodiment, the pharmaceutical formulations as described herein are administered to patients with an inflammatory condition. In certain embodiments the inflammatory condition is neurogenic inflammation. In another embodiment, the subject suffers from rheumatoid arthritis or a gastro-intestinal inflammatory disease.

In a further embodiment, the patient suffers from an inflammatory disorder. A subject suffering from an inflammatory disorder, for example, receives between about 1 to 100 U per treatment of a pharmaceutical formulation of the present invention. In a further example, the subject receives between about 1 to 10 U, 1 to 20 U, 1 to 30 U, 1 to 40 U, 1 to 50 U, 1 to 60 U, 1 to 70 U, 1 to 80 U, 1 to 90 U, 5 to 20 U, 5 to 30 U, 5 to 40 U, 5 to 50 U, 5 to 60 U, 5 to 70 U, 5 to 80 U, 5 to 90 U, or 5 to 100 U per treatment are administered to a patient with an inflammatory disorder. In a still further example, the subject receives about 1 U, about 10 U, about 20 U, about 30 U, about 40 U, about 50 U, about 60 U, about 70 U, about 80 U, about 90 U, or about 100 U per treatment are administered to a patient. Dosages greater than 100 U per treatment may also be administered to patients suffering from inflammation or an inflammatory disorder to achieve a therapeutic response.

### Skin Conditions

Methods for treating a skin disorder can have the step of local administration of a pharmaceutical formulation comprising a Clostridial derivative and a chemical depolarizing agent to a location of a skin disorder of a patient, such as to a face, hand or foot of a patient. The formulations can be locally administered with the Clostridial derivative in an amount of between about 10⁻³ units/kg of patient weight and about 35 units/kg of patient weight. For example, the neurotoxin is locally administered in an amount of between about 10⁻² U/kg and about 25 U/kg of patient weight. In a further example, the neurotoxin is administered in an amount of between about 10⁻¹ U/kg and about 15 U/kg. In one method within the scope of the present invention, the neurotoxin is locally administered in an amount of between about 1 U/kg and about 10 U/kg. In a clinical setting it can be advantageous to administer from 1 U to 3000 U of a neurotoxin, such as botulinum toxin type A or B, to a skin disorder location by topical application or by subdermal administration, to effectively treat the skin disorder.

Administration of Clostridium toxin derivatives can be carried out at multiple sites in the skin, wherein the sites of adjacent injections are separated by about 0.1 to 10 cm, or about 0.5 to about 5 cm, for example, by about 1.5 to about 3 cm. The toxins may be any of the botulinum toxins A, B, C, D, E, F or G. The amounts administered may vary between 0.1 and 1000 U, or about 1 to about 40, or from about 5 to about 10 U, depending on the manufacture's specifications, the class of the toxin and the mode of administration. The repeat time range for these administrations for maintenance of the desired change varies substantially according to the location of the injection, the condition to be adjusted and the condition of the patient. Thus, the repeat time may vary from about 1 week to about 50 weeks, however a common range is about 4 to about 25 weeks, or even about 12 weeks to about 16 weeks.

The distances between administrations, for example, injections, can vary from about 1 mm to about 10 cm, suitably from about 5 mm to about 5 cm, and more usually from about 1 cm to about 3 cm. Thus, for example botulinum A may be suitably administered by intradermal injection between about 0.1 to about 10 U at a separation of from about 0.5 to about 10 cm.

In another embodiment, methods for treating cutaneous disorders comprise the step of administering a pharmaceutical formulation of the present invention to a subject in need thereof in an amount sufficient to reduce a sebaceous or mucous secretion are provided. In further embodiments, the pharmaceutical formulations described herein are administered to a patient without producing muscle weakness. In certain embodiments the pharmaceutical formulations of the present invention are injected into one or more sites of an eyelid or conjunctiva. In another embodiment, the formulations of the present invention are administered to a body surface.

In another embodiment, the pharmaceutical formulations are administered in an amount sufficient to reduce cutaneous bacterial or fungal growth, including but not limited to Staphylococcus; Streptococcus and Moraxella. For example, the pharmaceutical formulations of the present invention are administered to an area selected from the group consisting of: eyelid; scalp; feet; groin; and armpit to reduce cutaneous infection.

Non-limiting embodiments are disclosed below:
1. A method of inducing local, partial or complete muscle denervation in a subject, comprising:

administering a therapeutically effective amount of a Clostridial toxin derivative; and

administering at least one chemical depolarizing agent;

wherein the at least one chemical depolarizing agent is administered within one week or 24 hours of administering the Clostridial toxin derivative.
2. The method of embodiment 1, wherein the Clostridial toxin derivative and depolarizing agent are administered at the same time.
3. The method of embodiment 1 or 2, wherein the Clostridial toxin derivative and depolarizing agent are administered in the same composition.
4. The method of any previous embodiment, wherein at least one of the at least one chemical depolarizing agents is selected from a potassium channel blocker, a calcium channel ionophore, sodium channel ionophore, and potassium.
5. The method of any previous embodiment, wherein at least one of the at least one chemical depolarizing agents is 4-aminopyridine (4-AP) or 3,4-diaminopyridine (DAP).
6. The method of any previous embodiment, wherein the Clostridial toxin derivative is a botulinum toxin.
7. The method of embodiment 6 wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.
8. The method of any previous embodiment, wherein administering the Clostridial toxin derivative comprises administering about 1-200 Units of the Clostridial toxin derivative.
9. The method of any previous embodiment, wherein administering the at least one chemical depolarizing agent increases the duration of effect of muscle denervation in the subject as compared to administering the Clostridial toxin derivative alone, wherein the at least one chemical depolarizing agent accelerates onset of the Clostridial toxin intoxication, and/or wherein the therapeutically effective amount of the Clostridial toxin derivative administered is lower than that of the Clostridial toxin derivative administered alone.
10. The method of embodiment 9, wherein the increase in duration of muscle denervation is at least about 50-200% longer as compared to administering the Clostridial toxin derivative alone.
11. The method of any previous embodiment, wherein the duration of the muscle denervation is at least about 1-14 days longer than the duration of muscle denervation when the Clostridial toxin derivative is administered alone.
12. The method of any previous embodiment, wherein at least one of the Clostridial toxin derivative and the depolarizing agent are locally administered.
13. The method of embodiment 12, wherein the local administration is by injection or topical application, or wherein the at least one of the Clostridial toxin derivative and the depolarizing agent are locally administered via dissolving microneedle patches.
14. The method of embodiment 13, wherein the injection is selected from the group consisting of non-intramuscular injection and subdermal injection.
15. The method of any previous embodiment, wherein inducing local, partial or complete muscle denervation is effective to treat a condition or symptom selected from the group consisting of a neuromuscular disease, pain, a urological disorder, inflammation, and skin disorders.
16. The method of any previous embodiment, wherein inducing local, partial or complete muscle denervation is used for cosmetically modifying soft-tissue features of the subject.
17. A method of inducing local, partial or complete muscle paralysis in a subject, comprising:

administering a therapeutically effective amount of a Clostridial toxin derivative; and

administering at least one chemical depolarizing agent;

wherein the at least one chemical depolarizing agent is administered within one week or 24 hours of administering the Clostridial toxin derivative.
18. The method of embodiment 17, wherein the toxin and depolarizing agent are administered at the same time.
19. The method of embodiment 17 or 18, wherein the Clostridial toxin derivative and depolarizing agent are administered in the same composition.
20. The method of any one of embodiments 17 to 19, wherein at least one of the at least one chemical depolarizing agents is selected from a potassium channel blocker, a calcium channel ionophore, a sodium channel ionophore, and potassium.
21. The method of any one of embodiments 17 to 20, wherein at least one of the at least one chemical depolarizing agents is 4-aminopyridine (4-AP) or 3,4-diaminopyridine (DAP).
22. The method of any one of embodiments 17 to 21, wherein the Clostridial toxin derivative is a botulinum toxin.
23. The method of embodiment 22, wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.
24. The method of any one of embodiments 17 to 23, wherein administering the Clostridial toxin derivative comprises administering about 1-200 Units of the Clostridial toxin derivative.
25. The method of any one of embodiments 17 to 24, wherein administering the at least one chemical depolarizing agent increases the duration of effect of muscle paralysis in the subject as compared to administering the Clostridial toxin derivative alone, wherein the at least one chemical depolarizing agent accelerates onset of the Clostridial toxin intoxication, and/or wherein the therapeutically effective amount of the Clostridial toxin derivative administered is lower than that of the Clostridial toxin derivative administered alone.
26. The method of embodiment 25, wherein the increase in duration of muscle paralysis is at least about 50-200% as compared to administering the Clostridial toxin derivative alone.
27. The method of any one of embodiments 17 to 26, wherein the duration of the muscle paralysis is at least about 1-14 days longer than the duration of muscle paralysis when the Clostridial toxin derivative is administered alone.
28. The method of any one of embodiments 17 to 27, wherein at least one of the Clostridial toxin derivative and the depolarizing agent are locally administered.
29. The method of embodiment 28, wherein the local administration is by injection or topical application, or wherein the at least one of the Clostridial toxin derivative and the depolarizing agent are locally administered via dissolving microneedle patches.
30. The method of embodiment 29, wherein the injection is selected from the group consisting of non-intramuscular injection and subdermal injection.
31. The method of any one of embodiments 17 to 30, wherein inducing local, partial or complete muscle paralysis is effective to treat a condition or symptom selected from the group consisting of a neuromuscular disease, pain, a urological disorder, inflammation, and skin disorders.
32. The method of any one of embodiments 17 to 31, wherein inducing local, partial or complete muscle paralysis is used for cosmetically modifying soft-tissue features of the subject.
33. A pharmaceutical preparation for use in inducing local, partial or complete muscle denervation in a subject, the preparation comprising:

a therapeutically effective amount of a Clostridial toxin derivative; and

at least one chemical depolarizing agent.
34. The preparation of embodiment 33, wherein at least one of the at least one chemical depolarizing agents is selected from a potassium channel blocker, a calcium channel ionophore, a sodium channel ionophore, and potassium.
35. The preparation of embodiment 33 or 34, wherein at least one of the at least one chemical depolarizing agents is 4-aminopyridine (4-AP) or 3,4-diaminopyridine (DAP).
36. The preparation of any one of embodiments 33 to 35, wherein the calcium channel ionophore is selected from the group consisting of ionmycin and calcimycin and the sodium channel ionophore is selected from the group consisting of monensin and gramecidin.
37. The preparation of any one of embodiments 33 to 36, wherein the Clostridial toxin derivative is a botulinum toxin.
38. The preparation of embodiment 37, wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.
39. The preparation of any one of embodiments 33 to 38, wherein the therapeutically effective amount of the Clostridial toxin derivative is about 1-200 Units.
40. The preparation of any one of embodiments 33 to 39, further comprising at least one stabilizer.
41. The preparation of embodiment 40, wherein the at least one stabilizer is selected from an albumin, a non-oxidizing amino acid derivative, a caprylate, a polysorbate, an amino acid, and a divalent metal cation.
42. A pharmaceutical preparation for use in inducing local, partial or complete muscle paralysis in a subject, the preparation comprising:

a therapeutically effective amount of a Clostridial toxin derivative; and

at least one chemical depolarizing agent.

43. The preparation of embodiment 42, wherein at least one of the at least one chemical depolarizing agents is selected from a potassium channel blocker, a calcium channel ionophore, a sodium channel ionophore, and potassium.
44. The preparation of embodiment 42 or 43, wherein at least one of the at least one chemical depolarizing agents is 4-aminopyridine (4-AP) or 3,4-diaminopyridine (DAP).
45. The preparation of embodiment 44, wherein the calcium channel ionophore is selected from the group consisting of ionmycin and calcimycin and the sodium channel ionophore is selected from the group consisting of monensin and gramecidin.
46. The preparation of any one of embodiments 42 to 45, wherein the Clostridial toxin derivative is a botulinum toxin.
47. The preparation of embodiment 46, wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.
48. The preparation of any one of embodiments 42 to 47, wherein the therapeutically effective amount of the Clostridial toxin derivative is about 1-200 Units.
49. The preparation of any one of embodiments 42 to 48, further comprising at least one stabilizer.
50. The preparation of embodiment 49, wherein the at least one stabilizer is selected from an albumin, a non-oxidizing amino acid derivative, a caprylate, a polysorbate, an amino acid, and a divalent metal cation.
51. A method of inducing local, partial or complete muscle denervation in a subject, comprising:

providing for administration a therapeutically effective amount of a Clostridial toxin derivative; and

providing for administration, or instructing to administer, a chemical depolarizing agent;

whereby administration of the chemical depolarizing agent within one week or 24 hours of administration of the Clostridial toxin derivative induces local, partial or complete muscle denervation in the subject.
52. A method of inducing local, partial or complete muscle paralysis in a subject, comprising:

providing for administration a therapeutically effective amount of a Clostridial toxin derivative; and

providing for administration, or instructing to administer, a chemical depolarizing agent;

whereby administration of the chemical depolarizing agent within 24 hours of administration of the Clostridial toxin derivative induces local, partial or complete muscle paralysis in the subject.
53. A method of inducing local, partial or complete muscle paralysis or muscle denervation in a subject, comprising:

providing for administration a therapeutically effective amount of a Clostridial toxin derivative; and

instructing administration of a chemical depolarizing agent within about one week or about 24 hours of administration of the Clostridial toxin derivative, whereby administration of the chemical depolarizing agent as instructed provides local, partial or complete muscle paralysis or muscle denervation in the subject.
54. A method of inducing local, partial or complete muscle paralysis or muscle denervation in a subject, comprising:

providing for administration a chemical depolarizing agent; and

instructing administration of a therapeutically effective amount of a Clostridial toxin derivative within about one week or within about 24 hours of administration of the chemical depolarizing agent, whereby administration of the Clostridial toxin derivative as instructed provides local, partial or complete muscle paralysis or muscle denervation in the subject.
55. The method of any of embodiments 1-3, 17-19, and 51-54, wherein the chemical depolarizing agent is 3,4-diaminopyridine (DAP).
56. The method of embodiment 55, wherein the Clostridial toxin derivative is BoNT/E.
57. The method of embodiment 55, wherein the method enhances neurotransmission followed by a faster muscle denervation in the subject as compared to administering the Clostridial toxin derivative alone.
58. The method of embodiment 55, wherein the therapeutically effective amount of the Clostridial toxin derivative administered is lower than that of the Clostridial toxin derivative administered alone.
59. The method of embodiment 56, wherein the method enhances neurotransmission followed by a faster muscle denervation in the subject as compared to administering BoNT/E alone.

60. The method of embodiment 56, wherein the therapeutically effective amount of the Clostridial toxin derivative administered is lower than that of BoNT/E administered alone.
61. The method of any of embodiments 55-60, wherein at least one of the Clostridial toxin derivative and DAP are locally administered.
62. The method of embodiment 61, wherein the local administration is by injection or topical application, or wherein the at least one of the Clostridial toxin derivative and the depolarizing agent are locally administered via dissolving microneedle patches.
63. The method of embodiment 62, wherein the injection is selected from the group consisting of non-intramuscular injection and subdermal injection.
64. The method of any of embodiments 55-63, wherein inducing local, partial or complete muscle denervation is effective to treat a condition or symptom selected from the group consisting of a neuromuscular disease, pain, a urological disorder, inflammation, and skin disorders.
65. The method of any of embodiments 55-64, wherein inducing local, partial or complete muscle denervation is used for cosmetically modifying soft-tissue features of the subject.
66. A pharmaceutical preparation, comprising:

a therapeutically effective amount of a Clostridial toxin derivative; and

at least one chemical depolarizing agent.
67. The preparation of embodiment 66, wherein at least one of the at least one chemical depolarizing agents is selected from a potassium channel blocker, a calcium channel ionophore, a sodium channel ionophore, and potassium.
68. The preparation of embodiment 66 or 67, wherein at least one of the at least one chemical depolarizing agents is 4-aminopyridine (4-AP) or 3,4-diaminopyridine (DAP).
69. The preparation of any one of embodiments 66 to 68, wherein the calcium channel ionophore is selected from the group consisting of ionmycin and calcimycin and the sodium channel ionophore is selected from the group consisting of monensin and gramecidin.
70. The preparation of any one of embodiments 66 to 69, wherein the Clostridial toxin derivative is a botulinum toxin.
71. The preparation of embodiment 70, wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.
72. The preparation of any one of embodiments 66 to 71, wherein the therapeutically effective amount of the Clostridial toxin derivative is about 1-200 Units.
73. The preparation of any one of embodiments 66 to 72, further comprising at least one stabilizer.
74. The preparation of embodiment 73, wherein the at least one stabilizer is selected from an albumin, a non-oxidizing amino acid derivative, a caprylate, a polysorbate, an amino acid, and a divalent metal cation.
75. The preparation of any of embodiments 33-50 and 66-74, wherein the preparation is not formed in situ by combining the Clostridial toxin derivative and the at least one chemical depolariaing agent in situ.
76. The preparation of any of embodiments 33-50 and 66-75, comprising no additional active ingredient.

### III. EXAMPLES

The following examples are illustrative in nature and are in no way intended to be limiting.

### Example 1

### Combination Therapy with A Botulinum Toxin Type A Neurotoxin and Chemical Depolarizing Agents

Sprague Dawley rats were intramuscularly injected into the tibialis anterior (TA) muscle with one of (i) 2.3 U/kg of botulinum toxin type A neurotoxin (BOTOX®) in 5 μL volume of normal saline (NS), or (ii) 50 μL 100 μM 4-AP. The rats were analyzed and received a digital abduction assay score on days 0-25 according to the method of Briode et al. (Toxicon, 2013, 71:18-24) with the results shown in FIG. 1.

### Example 2

### Combination Therapy with BoNT/A and 4-AP

Mouse hemidiaphragms from CD-1 mice were bathed in Krebs-Ringer buffer at 35 C gassed with 95% O₂ and 5% CO₂. Muscle contractions were elicited by electrical stimulation (0.2-ms pulses of 5V at 0.2 Hz) of the nerve. Three hundred micromolar 4-AP or normal saline was added to the bath followed by the addition of 30 pM BoNT/A (FIG. 2A). The 4-AP and BoNT/A were washed away after 20 minutes. Muscle twitch amplitude was plotted against minutes post treatment with 30 pM BoNT/A. The 4-AP increased the amplitude of muscle tension by approximately 2.5 fold and increased the onset of paralysis relative to the hemi-diaphragm that was treated with normal saline (FIG. 2A). A plot for the time required for 50% paralysis is shown in FIG. 2B, demonstrating approximately 25% faster BoNT/A onset with 4-AP (FIG. 2B).

### Example 3

### Combination Therapy with BoNT/E and DAP

Mouse hemidiaphragms from CD-1 mice were bathed in Krebs-Ringer buffer at 35 C gassed with 95% O₂ and 5% CO₂. Muscle contractions were elicited by electrical stimulation (0.2-ms pulses of 5V at 0.2 Hz) of the nerve. One hundred picomolar recombinant BoNT/E (FIG. 3A) or 10 pM recombinant BoNT/E (FIG. 3B) in vehicle or formulated in 50 μM DAP was added directly to the Krebs-Ringer buffer, and the time taken for 50% paralysis (TTP₅₀) or for 90% paralysis (TTP₉₀) was recorded. DAP enhanced neurotransmission and increased uptake of 100 pM BoNT/E (FIG. 3A) or 10 pM BoNT/E (FIG. 3B) as revealed by the faster time to TTP₅₀ and TTP₉₀.

The time to 50% paralysis (TTP₅₀) and 90% paralysis (TTP₉₀) for 100 pM BoNT/E in vehicle or 50 μM DAP is presented as bar graph (FIG. 3A). The relative time to 50% paralysis for 100 pM BoNT/E in vehicle or 50 μM DAP is also presented (FIG. 3A). The time to 50% paralysis (TTP₅₀) and 90% paralysis (TTP₉₀) for 10 pM BoNT/E in vehicle or 50 μM DAP is presented as bar graph (FIG. 3B). The relative time to 50% paralysis for 10 pM BoNT/E in vehicle or 50 μM DAP is presented (FIG. 3B). DAP-increased uptake of 100 pM and 10 pM BoNT/E resulted in faster onset of neuroparalysis in the mouse phrenic nerve hemidiaphragm.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method of inducing local, partial or complete muscle denervation or muscle paralysis in a subject, comprising:
administering a therapeutically effective amount of a Clostridial toxin derivative; and
administering at least one chemical depolarizing agent;
wherein the at least one chemical depolarizing agent is administered within one week or 24 hours of administering the Clostridial toxin derivative.

2. The method of claim 1, wherein the Clostridial toxin derivative and depolarizing agent are administered at the same time.

3. The method of claim 1 or 2, wherein the Clostridial toxin derivative and depolarizing agent are administered in the same composition.

4. The method of any previous claim, wherein at least one of the at least one chemical depolarizing agents is selected from a potassium channel blocker, a calcium channel ionophore, a sodium channel ionophore, and potassium.

5. The method of any previous claim, wherein at least one of the at least one chemical depolarizing agents is 4-aminopyridine (4-AP) or 3,4-diaminopyridine (DAP).

6. The method of any previous claim, wherein the Clostridial toxin derivative is a botulinum toxin.

7. The method of claim 6 wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.

8. The method of any previous claim, wherein administering the Clostridial toxin derivative comprises administering about 1-200 Units of the Clostridial toxin derivative.

9. The method of any previous claim, wherein administering the at least one chemical depolarizing agent increases the duration of effect of muscle denervation or paralysis in the subject as compared to administering the Clostridial toxin derivative alone, wherein the at least one chemical depolarizing agent accelerates onset of the Clostridial toxin intoxication, and/or wherein the therapeutically effective amount of the Clostridial toxin derivative administered is lower than that of the Clostridial toxin derivative administered alone.

10. The method of claim 9, wherein the increase in duration of muscle denervation or paralysis is at least about 50-200% longer as compared to administering the Clostridial toxin derivative alone.

11. The method of any previous claim, wherein the duration of the muscle denervation or paralysis is at least about 1-14 days longer than the duration of muscle denervation or paralysis when the Clostridial toxin derivative is administered alone.

12. The method of any previous claim, wherein at least one of the Clostridial toxin derivative and the depolarizing agent are locally administered.

13. The method of claim 12, wherein the local administration is by injection or topical application, or wherein the at least one of the Clostridial toxin derivative and the depolarizing agent are locally administered via dissolving microneedle patches.

14. The method of claim 13, wherein the injection is selected from the group consisting of non-intramuscular injection and subdermal injection.

15. The method of any previous claim, wherein inducing local, partial or complete muscle denervation or paralysis is effective to treat a condition or symptom selected from the group consisting of a neuromuscular disease, pain, a urological disorder, inflammation, and skin disorders.

16. The method of any previous claim, wherein inducing local, partial or complete muscle denervation or paralysis is used for cosmetically modifying soft-tissue features of the subject.

17. A pharmaceutical preparation, the preparation comprising:
a therapeutically effective amount of a Clostridial toxin derivative; and
at least one chemical depolarizing agent.

18. The preparation of claim 17, wherein at least one of the at least one chemical depolarizing agents is selected from a potassium channel blocker, a calcium channel ionophore, a sodium channel ionophore, and potassium.

19. The preparation of claim 17 or 18, wherein at least one of the at least one chemical depolarizing agents is 4-aminopyridine (4-AP) or 3,4-diaminopyridine (DAP).

20. The preparation of any one of claims 17 to 19, wherein the calcium channel ionophore is selected from the group consisting of ionmycin and calcimycin and the sodium channel ionophore is selected from the group consisting of monensin and gramecidin.

21. The preparation of any one of claims 17 to 20, wherein the Clostridial toxin derivative is a botulinum toxin.

22. The preparation of claim 21, wherein the botulinum toxin is selected from the group consisting of botulinum toxin types BoNT/A, BoNT/B, BoNT/C, BoNT/D, BoNT/E, BoNT/F, BoNT/G, BoNT/H, BoNT/X, eBoNT/J, and mosaic toxins selected from BoNT/DC, BoNT/CD, and BoNT/FA.

23. The preparation of any one of claims 17 to 22, wherein the therapeutically effective amount of the Clostridial toxin derivative is about 1-200 Units.

24. The preparation of any one of claims 17 to 23, further comprising at least one stabilizer.

25. The preparation of claim 24, wherein the at least one stabilizer is selected from an albumin, a non-oxidizing amino acid derivative, a caprylate, a polysorbate, an amino acid, and a divalent metal cation.

26. The method of any of claims 1-16 or the pharmaceutical preparation according to any of claims 17-25, wherein the chemical depolarizing agent is 3,4-diaminopyridine (DAP).

27. The method or pharmaceutical preparation of claim 26, wherein the Clostridial toxin derivative is BoNT/E.

28. The method of claim 26, wherein the method enhances neurotransmission followed by a faster muscle denervation or paralysis in the subject as compared to administering the Clostridial toxin derivative alone.

29. The method of claim 28, wherein the method enhances neurotransmission followed by a faster muscle denervation or paralysis in the subject as compared to administering BoNT/E alone.
